(19) United States
(12) Patent Application Publication
(10) Pub. No.: US 2008/0103141 A1
(43) Pub. Date: May 1, 2008

(54) New compounds

(75) Inventors: Peter Brandt, Uppsala (SE); Gary Johansson, Solna (SE); Lars Johansson, Bromma (SE); Tobias Koolmeister, Uppsala (SE); Bjorn Nilsson, Stockholm (SE); Teresa Sandvall, Stockholm (SE); Michael Weber, Kista (SE)

Correspondence Address: EDWARDS ANGELL PALMER & DODGE LLP, P.O. BOX 55874, BOSTON, MA, 02205, US

(73) Assignee: Biovitrum, Stockholm (SE)

(21) Appl. No.: 11/897,400

(22) Filed: Aug. 29, 2007

Related U.S. Application Data
(60) Provisional application No. 60/860,737, filed on Nov. 21, 2006.

(30) Foreign Application Priority Data
Aug. 30, 2006 (SE) 0601775-0

(51) Int. Cl. A61K 31/506; A61K 31/5377; A61P 3/00; C07D 239/34; C07D 413/14

(52) U.S. Cl. 514/235.8; 514/274; 544/123; 544/316

(57) ABSTRACT

The present invention relates to compounds of Formula (Ia) 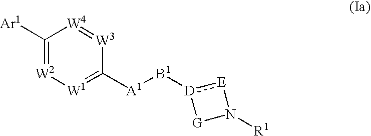
and pharmaceutically acceptable salts, hydrates, geometrical isomers, racemates, tautomers, optical isomers and N-oxides thereof, wherein W¹ and W³ are N and W² and W⁴ are CR¹², or W¹ and W³ are CR¹² and W² and W⁴ are N. The invention also relates to pharmaceutical compositions comprising these compounds, and to the use of these compounds for the prophylaxis and treatment of medical conditions relating to disorders of the G-protein-coupled receptor GPR119, such as diabetes and obesity.

### CROSS-REFERENCE TO RELATED APPLICATIONS

This application claims benefit of U.S. provisional application 60/860,737 filed Nov. 21, 2006, and Swedish application 0601775-0 filed Aug. 30, 2006, the entire contents of each which is herein incorporated by reference.

### FIELD OF INVENTION

The present invention relates to certain novel compounds, to pharmaceutical compositions comprising these novel compounds, and to the use of these compounds for the prophylaxis and treatment of medical conditions relating to disorders of the G-protein-coupled receptor GPR119 such as diabetes and obesity.

### BACKGROUND ART

Diabetes mellitus is a group of disorders characterized by abnormal glucose homeostasis resulting in high levels of blood glucose. The most common cases of diabetes mellitus are Type 1 (also referred to as insulin-dependent diabetes mellitus or IDDM) and Type 2 diabetes (also referred to as non-insulin-dependent diabetes mellitus or NIDDM). Type 2 diabetes accounts for approximately 90% of all diabetic cases. Type 2 diabetes is a serious progressive disease that results in the development of microvascular complications (e.g. retinopathy, neuropathy, nephropathy) as well as macrovascular complications (e.g. accelerated atherosclerosis, coronary heart disease, stroke). More than 75% of people with Type 2 diabetes die of cardiovascular diseases.

The increasing-prevalence of obesity together with an ageing population is contributing to the predicted explosion in diabetes across the globe. Current projections suggest that 300 million people worldwide have diabetes by 2025.

The pathogenesis of Type 2 diabetes involves insulin resistance, insulin secretory dysfunction (i.e. pancreatic beta cell dysfunction) and hepatic glucose overproduction. Insulin resistance is highly correlated with obesity. Accumulating reports suggest insulin resistance to be central to a cluster of metabolic abnormalities—including dyslipidemia, hypertension, endothelial dysfunction, reduced fibrinolysis, and chronic systemic inflammation—that together are responsible for the increased cardiovascular risk.

Current antidiabetic therapy is targeting the defects mentioned above. For instance, sulphonylureas increase production of endogenous insulin. However, this enhanced insulin production is not glucose dependent and there is risk for developing hypoglycaemia. Metformin lowers hepatic glucose output. Thiazolidindiones (TZDs) reduce insulin resistance in muscle and liver and suppress inflammatory responses. A major side effect of TZDs is weight gain due to fluid retention and increase in total body fat. An earlier drug in this class, troglitazone, was withdrawn due to rare but serious cases of hepatotoxicity. Current therapies have limited durability and/or significant side effects.

The widespread availability and increased consumption of Western diet combined with the adoption of a sedentary life-style has increased the number of obese people. Obesity is linked to a wide range of medical complications, such as diabetes, cardiovascular disease and cancer. In addition, being overweight can exacerbate the development of osteoporosis and asthma. Obesity is also proven to double the risk of hypertension. Obesity has only recently been regarded as a disease in the sense of being a specific target for medical therapy. Current therapies for obesity are based on diet and exercise and stomach surgery for extremely obese patients. Two weight loss medications are today available for long-term use. Sibutramine, a serotonin- and noradrenaline-reuptake inhibitor, controls appetite by producing a feeling of satiety. However, a prominent side effect is hypertension. Orlistat inhibits the lipase-mediated breakdown of fat in the gastrointestinal tract, thereby limiting caloric intake resulting in weight loss. However, approximately 20% of the patients using Orlistat develop faecal incontinence and urgency. Thus, there is an unmet medical need for new and novel antidiabetic and antiobesity therapies.

GPR119 (GenBank No. NM 178471) is a G-protein coupled receptor identified as SNORF25 in WO 00/50562. In humans, GPR119 is selectively expressed in pancreas and gastrointestinal tract. Activation of GPR119 by lysophosphatidylcholine (LPC) induces glucose-dependent insulin secretion from pancreatic beta-cells (Soga et al., Biochem. Biophys. Res. Commun. 326, 744-751, 2005). GPR119 agonists stimulate insulin secretion in rat islets and reduce blood glucose in diabetic Lepr^{db/db} mice (WO 2004/065380). Another endogenous ligand for GPR119, oleoylethanolamide (OEA), and a small molecule GPR119 agonist, PSN632408, both suppress food intake and reduce body weight gain in rat (Overton et al., Cell Metabolism 3, 167-175, 2006). Taken together, these data suggest that GPR119 is an interesting target for treating diabetes and/or obesity.

WO 2004/065380, WO 2004/076413, WO 2005/007647, WO 2005/007658 and WO 2005/121121 discloses compounds that are modulators of the Rup3 receptor, also referred to as SNORF25 (WO 00/50562) or as GPR119 (Fredriksson et al., FEBS Lett, 554, 381-388, 2003), and which inter alia may be used for the treatment of metabolic disorders and complications thereof, such as, diabetes and obesity.

WO 2005/061489, WO 2006/067531, WO 2006/067532 and WO 2006/070208 disclose compounds that are agonists of GPR116, also referred to as SNORF25 or as GPR119 (see Overton et al, Cell Metabolism 3, 167-175, 2006), and which inter alia may be used for the treatment of metabolic disorders and complications thereof, such as diabetes and obesity. WO 2006/076231 discloses a synergistic effect of a GPR119 agonist in combination with a DPP-IV inhibitor, in lowering elevated glucose levels in mice. Further, a synergistic effect with the said combination is shown in increasing blood GLP-1 levels after glucose challenge in mice.

### DISCLOSURE OF THE INVENTION

It has surprisingly been found that compounds of the general Formula (Ia) to (Id) are active as agonists of GPR119 and are potentially useful in the treatment or prophylaxis of disorders relating to GPR119. Examples of such disorders include Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

### DEFINITIONS

The following definitions shall apply throughout the specification and the appended claims.

Unless otherwise stated or indicated, the term “C₁₋₆-alkyl” denotes a straight or branched alkyl group having from 1 to 6 carbon atoms. For parts of the range “C₁₋₆-alkyl”, all subgroups thereof are contemplated, such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. Examples of said C₁₋₆-alkyl include methyl, ethyl, n-propyl, isopropyl, n-butyl, isobutyl, sec-butyl, t-butyl and straight- and branched-chain pentyl and hexyl.

Unless otherwise stated or indicated, the term “cyano-C₁₋₆-alkyl” denotes a C₁₋₆-alkyl group, as defined above, substituted with a cyano group. Exemplary cyano-C₁₋₆-alkyl groups include 2-cyanoethyl and 3-cyanopropyl.

Unless otherwise stated or indicated, the term “amino-C₁₋₆-alkyl” denotes a C₁₋₆-alkyl group, as defined above, substituted with an amino group. Exemplary amino-C₁₋₆-alkyl groups include 2-aminoethyl and 3-aminopropyl.

Unless otherwise stated or indicated, the term “hydroxy-C₁₋₆-alkyl” denotes a straight or branched alkyl group that has a hydrogen atom thereof replaced with OH. Examples of said hydroxy-C₁₋₆-alkyl include hydroxymethyl, 2-hydroxyethyl, 2-hydroxypropyl, 3-hydroxy-3-methylbutyl, 2-hydroxybutyl and 2-hydroxy-2-methylpropyl. Derived expressions such as “C₁₋₆-alkoxy”, “C₁₋₆-alkylthio” and “C₁₋₆-alkylamino” are to be construed accordingly where an C₁₋₆-alkyl group is attached to the remainder of the molecule through an oxygen, sulfur or nitrogen atom, respectively. For parts of the range “C₁₋₆-alkoxy” all subgroups thereof are contemplated such as C₁₋₄-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc. Examples of said “C₁₋₆-alkoxy” include methoxy, ethoxy, n-propoxy, isopropoxy, n-butoxy, isobutoxy, sec-butoxy, t-butoxy and straight- and branched-chain pentoxy and hexoxy etc. Subgroups of “C₁₋₆-alkylthio” and “C₁₋₆-alkylamino” are to be construed accordingly.

Unless otherwise stated or indicated, the term “C₁₋₄-alkylsulfinyl” denotes a group C₁₋₄-alkyl-S(O)—. Exemplary C₁₋₄-alkylsulfinyl groups include methylsulfinyl and ethylsulfinyl. Unless otherwise stated or indicated, the term “dihydroxy-C₂₋₆-alkyl” denotes a C₂₋₆-alkyl group which is disubstituted with hydroxy and wherein said hydroxy groups are attached to different carbon atoms. Exemplary dihydroxy-C₂₋₆-alkyl groups include 2,3-dihydroxy-propyl and 2,4-dihydroxybutyl.

Unless otherwise stated or indicated, the term “di(C₁₋₄-alkyl)amino” denotes a group (C₁₋₄-alkyl)₂N—, wherein the two alkyl portions may be the same or different. Exemplary di(C₁₋₄-alkyl)amino groups include N,N-dimethylamino, N-ethyl-N-methylamino and N,N-diethylamino.

Unless otherwise stated or indicated, the term “di(C₁₋₄-alkyl)amino-C₂₋₄-alkyl” denotes a group di(C₁₋₄-alkyl)amino, as defined above, attached to a C₂₋₄-alkyl group. Exemplary di(C₁₋₄-alkyl)amino-C₂₋₄-alkyl groups include 2-(dimethylamino)ethyl and 3-(diethyl-amino)propyl.

Unless otherwise stated or indicated, the term “fluoro-C₁₋₆-alkyl” denotes a C₁₋₆-alkyl group substituted by one or more fluorine atoms. Examples of said fluoro-C₁₋₆-alkyl include 2-fluoroethyl, fluoromethyl, 2-fluoro-1-(fluoromethyl)ethyl, trifluoromethyl, 3,3,3-trifluoropropyl and 2,2,2-trifluoroethyl. Likewise, “aryl-C₁₋₆-alkyl” means a C₁₋₆-alkyl group substituted by an aryl group. Examples include benzyl, 2-phenylethyl, 1-phenylethyl and 2-methyl-2-phenylpropyl.

Unless otherwise stated or indicated, the term “arylcarbonyl-C₁₋₄-alkyl” denotes an arylcarbonyl group (e.g., benzoyl) that is attached through a C₁₋₄-alkyl group. Examples of said arylcarbonyl-C₁₋₄-alkyl include 3-oxo-3-phenylpropyl, 2-oxo-2-phenylethyl and 1-methyl-3-oxo-3-phenylpropyl.

Unless otherwise stated or indicated, the term “heteroarylcarbonyl-C₁₋₄-alkyl” denotes a heteroarylcarbonyl group (e.g., 3-pyridinylcarbonyl) that is attached through a C₁₋₄-alkyl group. Examples of said heteroarylcarbonyl-C₁₋₄-alkyl include 3-oxo-3-(3-pyridinyl)-propyl, 2-oxo-2-(3-pyridinyl)ethyl and 1-methyl-3-oxo-3-(3-pyridinyl)propyl.

Unless otherwise stated or indicated, the term “C₁₋₆-alkoxy-C₂₋₆-alkyl” denotes a straight or branched alkoxy group having from 1 to 6 carbon atoms connected to an alkyl group having from 2 to 6 carbon atoms. Examples of said C₁₋₆-alkoxy-C₂₋₆-alkyl include methoxyethyl, ethoxyethyl, isopropoxyethyl, n-butoxyethyl, t-butoxyethyl and straight- and branched-chain pentoxyethyl. For parts of the range “C₁₋₆-alkoxy-C₂₋₆-alkyl” all subgroups thereof are contemplated such as C₁₋₅-alkoxy-C₂₋₆-alkyl, C₁₋₄-alkoxy-C₂₋₆-alkyl, C₁₋₃-alkoxy-C₂₋₆-alkyl, C₁₋₂-alkoxy-C₂₋₆-alkyl, C₂₋₆-alkoxy-C₂₋₆-alkyl, C₂₋₅-alkoxy-C₂₋₆-alkyl, C₂₋₄-alkoxy-C₂₋₆-alkyl, C₂₋₃-alkoxy-C₂₋₆-alkyl, C₃₋₆-alkoxy-C₂₋₆-alkyl, C₄₋₅-alkoxy-C₂₋₆-alkyl, C₁₋₆-alkoxy-C₂₋₅-alkyl, C₁₋₆-alkoxy-C₂₋₄-alkyl, etc.

Unless otherwise stated or indicated, the term “C₂₋₆-alkenyl” denotes a straight or branched hydrocarbon chain radical containing one carbon-carbon double bond and having from 2 to 6 carbon atoms. Examples of said C₂₋₆-alkenyl include vinyl, allyl, 2,3-dimethylallyl, 1-butenyl, 1-pentenyl, and 1-hexenyl. For parts of the range “C₂₋₆-alkenyl”, all subgroups thereof are contemplated such as C₂₋₅-alkenyl, C₂₋₄-alkenyl, C₂₋₃-alkenyl, C₃₋₆-alkenyl, C₄₋₅-alkenyl, etc. Likewise, “aryl-C₂₋₆-alkenyl” means a C₂₋₆-alkenyl group substituted by an aryl group. Examples of said aryl-C₂₋₆-alkenyl include styryl and cinnamyl.

Unless otherwise stated or indicated, the term “C₂₋₆-alkynyl” denotes a straight or branched hydrocarbon chain radical containing one carbon-carbon triple bond and having from 2 to 6 carbon atoms. Examples of said C₂₋₆-alkynyl include ethynyl, 1-propynyl, 2-propynyl, 1-butynyl, 2-butynyl, and 1-methylprop-2-yn-1-yl. Likewise, aryl-C₂₋₆-alkynyl means a C₂₋₆-alkynyl group substituted by an aryl group. Examples of said aryl-C₂₋₆-alkynyl include phenylethynyl, 3-phenyl-1-propyn-1-yl, 3-phenyl-2-propyn-1-yl and 4-phenyl-2-butyn-1-yl.

The term “oxo” denotes

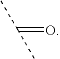

Unless otherwise stated or indicated, the term “C₃₋₇-cycloalkyl” denotes a cyclic alkyl group having a ring size from 3 to 7 carbon atoms and includes cyclopropyl, cyclobutyl, cyclopentyl, cyclohexyl, and cycloheptyl. For parts of the range “C₃₋₇-cycloalkyl” all subgroups thereof are contemplated such as C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₆₋₇-cycloalkyl.

Unless otherwise stated or indicated, the term “C₃₋₇-cycloalkyl-C₁₋₄-alkyl” denotes a C₃₋₇-cycloalkyl group attached to a C₁₋₄-alkyl group. Exemplary C₃₋₇-cycloalkyl-C₁₋₄-alkyl groups include cyclopropylmethyl, 1-cyclopropylethyl, cyclohexylmethyl and 2-cyclo-hexylethyl. When the cycloalkyl portion as part of the group C₃₋₇-cycloalkyl-C₁₋₄-alkyl is substituted with methyl, examples of such groups include (1-methylcyclopropyl)methyl and 2-(4-methylcyclohexyl)ethyl.

Unless otherwise stated or indicated, the term “C₇₋₈-bicyclyl” denotes a carbobicyclic saturated aliphatic ring system in which two non-adjacent carbon atoms of a monocyclic ring are linked by an alkylene bridge of between one and three additional carbon atoms. Examples of said C₇₋₈-bicyclyl include radicals obtainable from bicyclo[3.1.1]heptane, bicyclo[2.2.1]heptane (norbornane) and bicyclo[2.2.2]octane. Unless otherwise stated or indicated, the term C₇₋₈-bicyclylalkyl means a C₁₋₆-alkyl group substituted by a C₇₋₈-bicyclyl group as defined above. An exemplary C₇₋₈-bicyclylalkyl group is bicyclo[2.2.1]hept-2-ylmethyl (2-norbonylmethyl).

Unless otherwise stated or indicated, the term “C₅₋₈-cycloalkenyl” denotes a monocyclic or bicyclic alkenyl group of 5 to 8 carbon atoms having one carbon-carbon double bond. Examples of monocyclic cycloalkenyl groups are cyclopent-3-en-1-yl and cyclohexen-1-yl. An exemplary bicyclic cycloalkenyl group is bicyclo[2.2.1]hept-5-en-2-yl (norbornen-2-yl).

Unless otherwise stated or indicated, the term “oxo-C₄₋₆-cycloalkyl” refers to a C₄₋₆-cycloalkyl wherein one of the ring carbons is a carbonyl. Examples of “oxo-C₄₋₆-cycloalkyl” include 2-oxocyclobutyl, 3-oxocyclobutyl, 2-oxocyclopentyl and 4-oxo-cyclohexyl.

Unless otherwise stated or indicated, the term “fluoro-C₃₋₆-cycloalkyl” denotes a C₃₋₆-cycloalkyl group substituted by one or two fluorine atoms. Examples of said “fluoro-C₃₋₆-cycloalkyl” include 2,2-difluorocyclopropyl and 4-fluorocyclohexyl.

Unless otherwise stated or indicated, the term “C₁₋₃-alkoxy-C₄₋₆-cycloalkyl” denotes a C₄₋₆-cycloalkyl group substituted by a C₁₋₃-alkoxy group. Examples of said “C₁₋₃-alkoxy-C₄₋₆-cycloalkyl” include 4-methoxycyclohexyl and 2-ethoxycyclopentyl.

Unless otherwise stated or indicated, the term “methyl-C₃₋₆-cycloalkyl” denotes a C₃₋₆-cycloalkyl group substituted by one or two methyl groups. Examples of said “methyl-C₃₋₆-cycloalkyl” include 4-methylcyclohexyl and 3,3-dimethylcyclopentyl.

Unless otherwise stated or indicated, the term “acyl”, which may be straight or branched, denotes a carbonyl group that is attached through its carbon atom to a hydrogen atom to form a C₁-acyl group (i.e., a formyl group) or to an alkyl group, where alkyl is defined as above. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc. Exemplary acyl groups include formyl, acetyl (i.e., C₂-acyl), propanoyl, butanoyl, pentanoyl, hexanoyl.

Unless otherwise stated or indicated, the term “C₂₋₆-acyl-C₁₋₆-alkyl” refers to a group C₁₋₅-alkyl-(C═O)—C₁₋₆-alkyl. Exemplary C₂₋₆-acyl-C₁₋₆-alkyl groups include 2-acetylethyl and 3-acetylpropyl.

Unless otherwise stated or indicated, the term “C₁₋₆-alkylsulfonyl”, which may be straight or branched, denotes a hydrocarbon having from 1 to 6 carbon atoms with a sulfonyl group. For parts of the range “C₁₋₆-alkylsulfonyl” all subgroups thereof are contemplated such as C₁₋₅-alkylsulfonyl, C₁₋₄-alkylsulfonyl, C₁₋₃-alkylsulfonyl, C₁₋₂-alkylsulfonyl, C₂₋₆-alkylsulfonyl, C₂₋₅-alkylsulfonyl, C₂₋₄-alkylsulfonyl, C₂₋₃-alkylsulfonyl, C₃₋₆-alkylsulfonyl, C₄₋₅-alkylsulfonyl, etc. Exemplary C₁₋₆-alkylsulfonyl groups include methylsulfonyl, ethylsulfonyl, propylsulfonyl, n-butylsulfonyl, sec-butylsulfonyl, tert-butylsulfonyl, pentylsulfonyl and hexylsulfonyl.

Unless otherwise stated or indicated, the term “hydroxy-C₂₋₄-alkylsulfonyl” denotes a C₂₋₄-alkylsulfonyl group as defined above substituted with a hydroxy group. Examples of said hydroxy-C₂₋₄-alkylsulfonyl include hydroxymethylsulfonyl and 2-hydroxyethylsulfonyl.

Unless otherwise stated or indicated, the term “C₁₋₄-alkylsulfonamido” denotes a group C₁₋₄-alkyl-SO₂NH—. Exemplary C₁₋₄-alkylsulfonamido groups include methylsulfonyl-amino and ethylsulfonylamino.

Unless otherwise stated or indicated, the term “C₁₋₃-alkylene” refers to the diradicals methylene (—CH₂—), ethylene (—CH₂—CH₂—) and propylene (—CH₂—CH₂—CH₂—). In case the group denoted by E in Formula (Ia) forms a double bond with D, then E is a trivalent radical selected from (═CH₂—CH₂—) and (═CH₂—CH₂—CH₂—).

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring, preferably mono- or bicyclic. Examples of aryls are phenyl, indenyl, 2,3-dihydroindenyl (indanyl), 1-naphthyl, 2-naphthyl or 1,2,3,4-tetrahydronaphthyl.

Unless otherwise stated or indicated, the term “heteroaryl” refers to a mono- or bicyclic heteroaromatic ring system having 5 to 10 ring atoms in which one or more of the ring atoms are other than carbon, such as nitrogen, sulphur or oxygen. Only one ring need be aromatic and said heteroaryl moiety can be linked to the remainder of the molecule via a carbon or nitrogen atom in any ring. Examples of heteroaryl groups include furyl, pyrrolyl, thienyl, oxazolyl, isoxazolyl, imidazolyl, thiazolyl, isothiazolyl, pyridyl, pyrimidinyl, quinazolinyl, indolyl, isoindolyl, 1,3-dihydro-isoindolyl, pyrazolyl, pyridazinyl, quinolinyl, quinoxalinyl, thiadiazolyl, benzofuranyl, 2,3-dihydrobenzofuranyl, 1,3-benzodioxolyl, 1,4-benzodioxinyl, 2,3-dihydro-1,4-benzodioxinyl, benzothiazolyl, benzimidazolyl, benzothiadiazolyl, benzotriazolyl, indolinyl, isoindolinyl, and chromanyl groups.

Unless otherwise stated or indicated, the term “heterocyclyl” or “heterocyclic ring” refers to a non-aromatic fully saturated or partially unsaturated monocyclic ring system having 4 to 7 ring atoms with at least one heteroatom such as O, N, or S, and the remaining ring atoms are carbon. Examples of heterocyclic groups include piperidinyl, tetrahydropyranyl, tetrahydrofuranyl, oxetanyl, azepinyl, azetidinyl, pyrrolidinyl, morpholinyl, imidazolinyl, imidazolidinyl, thiomorpholinyl, pyranyl, dioxanyl, piperazinyl and 5,6-dihydro-4H-1,3-oxazin-2-yl. When present, the sulfur atom may be in an oxidized form (i.e., S═O or O═S═O). Exemplary heterocyclic groups containing sulfur in oxidized form are 1,1-dioxido-thiomorpholinyl and 1,1-dioxido-isothiazolidinyl.

When two groups R⁵, two groups R^{5A}, two groups R⁹ or two groups R^{9A} described herein form a heterocyclic ring and said heterocyclic ring is substituted with one or two oxo groups, examples of such groups include 2-pyrrolidon-1-yl, 2-piperidon-1-yl, 2-azetidinon-1-yl, 2,5-dioxopyrrolidin-1-yl and hydantoin-1-yl (i.e., 2,5-dioxoimidazolidin-1-yl).

When two groups R⁵, two groups R^{5A}, two groups R⁹ or two groups R^{9A} described herein form a heterocyclic ring and said heterocyclic ring is substituted with one or two fluoro atoms, examples of such groups include 4-fluoropiperidin-1-yl, 4,4-difluoropiperidin-1-yl, 3-fluoropyrrolidin-1-yl and 3,3-difluoropyrrolin-1-yl.

When two groups R⁵, two groups R^{5A}, two groups R⁹ or two groups R^{9A} described herein form a heterocyclic ring and said heterocyclic ring is substituted with hydroxy, examples of such groups include 4-hydroxypiperidin-1-yl, 3-hydroxypiperidin-1-yl, and 3-hydroxy-pyrrolidin-1-yl.

When two groups R⁵, two groups R^{5A}, two groups R⁹ or two groups R^{9A} described herein form a heterocyclic ring and said heterocyclic ring is substituted with amino, examples of such groups include 4-aminopiperidin-1-yl, 3-aminopiperidin-1-yl, and 3-aminopyrrolidin-1-yl.

When two groups R⁵, two groups R^{5A}, two groups R⁹ or two groups R^{9A} described herein form a heterocyclic ring and said heterocyclic ring is substituted with hydroxymethyl, examples of such groups include 2-(hydroxymethyl)pyrrolidin-1-yl, 2-(hydroxymethyl)-morpholin-4-yl and 4-(hydroxymethyl)piperidin-1-yl.

When two groups R⁵, two groups R^{5A}, two groups R⁹ or two groups R^{9A} described herein form a heterocyclic ring and said heterocyclic ring is substituted with methylamino or dimethylamino, examples of such groups include 3-dimethylaminopyrrolidin-1-yl and 3-methylaminopyrrolidin-1-yl.

Unless otherwise stated or indicated, the term “heteroaryl-C₁₋₄-alkyl” denotes a heteroaryl group that is attached through a C₁₋₄-alkyl group. Examples of said heteroaryl-C₁₋₄-alkyl include 2-(pyridin-2-yl)ethyl and 1,3 benzodioxol-5-ylmethyl.

“C-heterocyclyl” indicates bonding via a carbon atom of said heterocyclyl, for example piperidin-4-yl, tetrahydrofuran-2-yl, oxetan-3-yl, tetrahydrofuran-3-yl and 5,6-dihydro-4H-1,3-oxazin-2-yl, while “N-heterocyclyl” indicates bonding through nitrogen in a nitrogen-containing heterocyclyl group, for example piperidin-1-yl and piperazin-1-yl. When C-heterocyclyl is substituted by C₁₋₄-alkyl, said C₁₋₄-alkyl is attached to a ring nitrogen atom or a ring carbon atom thereof. Exemplary C-heterocyclyl groups substituted by C₁₋₄-alkyl include 1-methylpiperidin-4-yl and 3-methyloxetan-3-yl.

Unless otherwise stated or indicated, the term “N-heterocyclyl-C₂₋₄-alkyl” refers to a nitrogen-containing heterocyclyl group that is directly linked to a C₂₋₄-alkyl group via a nitrogen atom of said heterocyclyl. Exemplary N-heterocyclyl-C₂₋₄-alkyl groups include 2-(pyrrolidin-1-yl)ethyl, 3-(4-morpholinyl)propyl, 2-(piperazin-1-yl)ethyl and 2-(4-morpholinyl)ethyl.

When heterocyclyl as part of the group N-heterocyclyl-C₂₋₄-alkyl is substituted by methyl, said heterocyclyl is selected from 1-piperazinyl or 1-homopiperazinyl and said methyl is attached to the 4-position of the piperazine or homopiperazine ring. Exemplary N-heterocyclyl-C₂₋₄-alkyl groups wherein heterocyclyl is substituted with methyl are 2-(4-methylpiperazin-1-yl)ethyl, 2-(4-methylhomopiperazin-1-yl)ethyl.

Unless otherwise stated or indicated, the term “C-heterocyclyl-C₁₋₄-alkyl” refers to a heterocyclyl group that is directly linked to a C₁₋₄-alkyl group via a carbon atom of said heterocyclyl. Exemplary C-heterocyclyl-C₁₋₄-alkyl groups include tetrahydropyran-4-ylmethyl, piperidin-4-ylmethyl, tetrahydrofuran-2-ylmethyl, oxetan-3-ylmethyl and 2-(piperidinyl-4-yl)ethyl.

When heterocyclyl as part of the group C-heterocyclyl-C₁₋₄-alkyl is substituted by methyl, said methyl is attached to a ring nitrogen atom or ring carbon atom thereof. Exemplary C-heterocyclyl-C₁₋₄-alkyl groups wherein heterocyclyl is substituted with methyl are 2-(1-methylpiperidin-4-yl)ethyl and 3-methyloxetan-3-ylmethyl.

Unless otherwise stated or indicated, the term “oxo-N-heterocyclyl” denotes a nitrogen-containing heterocyclyl group that is substituted with one or two oxo groups.

Unless otherwise stated or indicated, the term “oxo-N-heterocyclyl-C₂₋₄-alkyl” refers to an oxo-N-heterocyclyl group that is directly linked to a C₂₋₄-alkyl group through a nitrogen atom of its heterocyclyl portion and where oxo-N-heterocyclyl is as defined above. Exemplary oxo-N-heterocyclyl-C₂₋₄-alkyl groups include 2-(2-pyrrolidon-1-yl)ethyl, 3-(2-pyrrolidon-1-yl)propyl and 2-(2,5-dioxoimidazolidin-1-yl)ethyl.

Unless otherwise stated or indicated, the term “fluoro-N-heterocyclyl” denotes a nitrogen-containing heterocyclyl group that is substituted at a position other than alpha to a ring heteroatom with one or two fluorine atoms.

Unless otherwise stated or indicated, the term “fluoro-N-heterocyclyl-C₂₋₄-alkyl” refers to a fluoro-N-heterocyclyl group that is directly linked to a C₂₋₄-alkyl group through a nitrogen atom of its heterocyclyl portion and where fluoro-N-heterocyclyl is as defined above. Exemplary fluoro-N-heterocyclyl-C₂₋₄-alkyl groups include 2-(3-fluoropyrrolidin-1-yl)-ethyl and 3-(3-fluoropyrrolidin-1-yl)propyl.

Unless otherwise stated or indicated, the term “hydroxy-N-heterocyclyl” denotes a nitrogen-containing heterocyclyl group that is substituted at a position other than alpha to a ring heteroatom with a hydroxy group.

Unless otherwise stated or indicated, the term “hydroxy-N-heterocyclyl-C₂₋₄-alkyl” refers to a hydroxy-N-heterocyclyl group that is directly linked to a C₂₋₄-alkyl group through a nitrogen atom of its heterocyclyl portion and where hydroxy-N-heterocyclyl is as defined above. Exemplary hydroxy-N-heterocyclyl-C₂₋₄-alkyl groups include 2-(4-hydroxy-piperidin-1-yl)ethyl and 3-(3-hydroxypiperidin-1-yl)propyl.

Unless otherwise stated or indicated, the term “amino-N-heterocyclyl” denotes a nitrogen-containing heterocyclyl group that is substituted at a position other than alpha to a ring heteroatom with an amino group.

Unless otherwise stated or indicated, the term “amino-N-heterocyclyl-C₂₋₄-alkyl” refers to a amino-N-heterocyclyl group that is directly linked to a C₂₋₄-alkyl group through a nitrogen atom of its heterocyclyl portion and where amino-N-heterocyclyl is as defined above. Exemplary amino-N-heterocyclyl-C₂₋₄-alkyl groups include 2-(4-aminopiperidin-1-yl)ethyl and 3-(3-aminopiperidin-1-yl)propyl.

Unless otherwise stated or indicated, the term “azabicyclyl” denotes a bicyclic heterocyclyl group with seven or eight atoms (including bridgehead atoms), wherein at least one ring member is a nitrogen atom and the remainder ring atoms being carbon. The said azabicyclyl may optionally contain a carbon-carbon double bond. Examples of azabicyclyl groups include carbon radicals obtainable from 1-azabicyclo[2.2.2]octane, 1-aza-bicyclo[2.2.1]heptane and azabicyclo[2.2.2]oct-2-ene.

“C-heterocyclylsulfonyl” refers to a heterocyclyl group that is directly bonded to SO₂ via a carbon atom. Exemplary C-heterocyclylsulfonyl groups include 4-piperidinylsulfonyl and tetrahydropyran-4-ylsulfonyl.

When C-heterocyclylsulfonyl is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclylsulfonyl group substituted by C₁₋₄-alkyl includes 1-methylpiperidin-4-ylsulfonyl.

Unless otherwise stated or indicated, the term “C₂₋₄-acylamino” denotes a group R^{b}(C═O)NH— wherein R^{b} is selected from C₁₋₃-alkyl. Exemplary C₂₋₄-acylamino groups include acetylamino and propionylamino.

Unless otherwise stated or indicated, the term “C₂₋₄-acylamino-C₁₋₄-alkyl” denotes a C₂₋₄ acylamino group, as defined above, attached to a C₁₋₄-alkyl group. Exemplary C₂₋₄-acylamino-C₁₋₄-alkyl groups include (acetylamino)methyl and 2-(acetylamino)ethyl.

Unless otherwise stated or indicated, the term “aminocarbonyl” refers to the radical NH₂(C═O)—.

Unless otherwise stated or indicated, the term “aminocarbonyl-C₁₋₄-alkyl” denotes a C₁₋₄-alkyl group, as defined above, substituted with an aminocarbonyl group. Exemplary aminocarbonyl-C₁₋₄-alkyl groups include 2-(aminocarbonyl)ethyl and 3-(aminocarbonyl)-propyl.

Unless otherwise stated or indicated, the term “carboxy” denotes a group —C(O)OH.

Unless otherwise stated or indicated, the term “carboxy-C₁₋₃-alkyl” refers to a carboxy group, as defined above, attached to a C₁₋₃-alkyl group. Exemplary carboxy-C₁₋₃-alkyl groups include 2-carboxyethyl and 3-carboxypropyl.

Unless otherwise stated or indicated, the term “carboxy-C₁₋₃-alkylcarbonylamino” refers to a carboxy-C₁₋₃-alkyl groups, as defined above, attached to the carbonyl carbon of carbonylamino (i.e., —C(O)NH—). Exemplary carboxy-C₁₋₃-alkylcarbonylamino groups include (2-carboxyethyl)carbonylamino and (3-carboxypropyl)carbonylamino.

“C-heterocyclylcarbonyl” refers to a heterocyclyl group that is directly bonded to a carbonyl group via a carbon atom while “N-heterocyclylcarbonyl” refers to a nitrogen-containing heterocyclyl group that is directly bonded to a carbonyl group via a nitrogen atom. Examples of N-heterocyclylcarbonyl groups include 1-piperidinylcarbonyl, 1-piperazinylcarbonyl and 1-pyrrolidincarbonyl. Exemplary C-heterocyclylcarbonyl groups include 3-piperidinylcarbonyl, 4-piperidinylcarbonyl and tetrahydropyranyl-4-ylcarbonyl.

When C-heterocyclylcarbonyl is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclylcarbonyl group substituted by C₁₋₄-alkyl includes 1-methylpiperidin-4-ylcarbbnyl.

The term “N-heterocyclylcarbonyl-C₂₋₄-alkyl” refers to a N-heterocyclylcarbonyl group that is directly linked to a C₂₋₄-alkyl group through its carbonyl carbon atom and where N-heterocyclylcarbonyl is as defined above. Exemplary N-heterocyclylcarbonyl-C₂₋₄-alkyl groups include 2-(pyrrolidin-1-ylcarbonyl)ethyl, 2-(piperazin-1-ylcarbonyl)ethyl and 2-(piperidin-1-ylcarbonyl)ethyl.

When heterocyclyl as part of the group N-heterocyclylcarbonyl-C₂₋₄-alkyl is substituted by methyl, said heterocyclyl is selected from 1-piperazinyl or 1-homopiperazinyl and said methyl is attached to the 4-position of the piperazine or homopiperazine ring. Exemplary N-heterocyclylcarbonyl-C₂₋₄-alkyl groups wherein heterocyclyl is substituted with methyl are 2-(4-methylpiperazin-1-ylcarbonyl)ethyl, 2-(4-methylhomopiperazin-1-ylcarbonyl)-ethyl.

The term “C-heterocyclylcarbonyl-C₂₋₄-alkyl” refers to a C-heterocyclylcarbonyl group that is directly linked to a C₂₋₄-alkyl group through its carbonyl carbon atom and where C-heterocyclylcarbonyl is as defined above. Exemplary C-heterocyclylcarbonyl-C₂₋₄-alkyl groups include 2-(tetrahydropyran-4-ylcarbonyl)ethyl, 2-(piperidin-3-ylcarbonyl)ethyl and 2-(piperidin-4-ylcarbonyl)ethyl.

When heterocyclyl as part of the group C-heterocyclylcarbonyl-C₂₋₄-alkyl is substituted by methyl, said heterocyclyl is selected from a nitrogen-containing heterocyclyl and said methyl is attached to a ring nitrogen atom thereof. An exemplary C-heterocyclylcarbonyl-C₂₋₄-alkyl group wherein heterocyclyl is substituted with methyl is 2-(1-methylpiperidin-4-ylcarbonyl)ethyl.

The term “C-heterocyclyloxy” refers to a heterocyclic group that is directly bonded to an oxygen atom via a carbon atom. Examples of C-heterocyclyloxy groups include 3-piperidinyloxy, 4-piperidinyloxy, 3-tetrahydrofuranyloxy, and 4-tetrahydropyranyloxy. When C-heterocyclyloxy is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclyloxy group substituted by C₁₋₄-alkyl includes 1-methylpiperidin-4-yloxy.

The term “hydroxy-C₂₋₄-alkoxy-C₁₋₄-alkyl” refers to a hydroxy-C₂₋₄-alkoxy group that is directly attached to a C₁₋₄-alkyl group. Representative examples of such groups include:

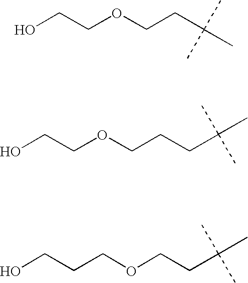

The term “phosphonooxy” refers to a group with the following chemical structure:

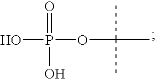

The term “amidino” refers to a group with the following chemical structure:

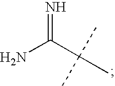

The term “guanidino” refers to a group with the following chemical structure:

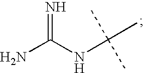

The chemical formula —C(OH)CH₃CF₃ refers to a group with the following chemical structure:

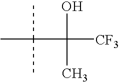

The term [C(OH)CH₃CF₃]—C₁₋₆-alkyl refers to a —C(OH)CH₃CF₃ group that is directly attached to a C₁₋₆-alkyl group. Representative examples of such groups include:

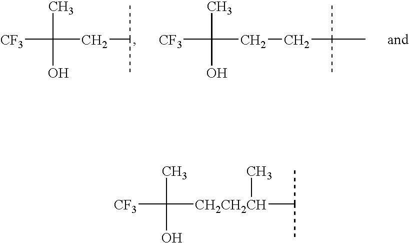

The chemical formula CF₃SO₃ refers to a group with the following chemical structure:

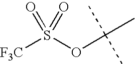

The carbon-carbon double or triple bonds present in the groups C₃₋₆-alkenyl, C₃₋₆-alkynyl, aryl-C₃₋₆-alkenyl and aryl-C₃₋₆-alkynyl as values for R² are meant to be located at positions other than conjugated with a carbonyl group or adjacent to a nitrogen, oxygen or sulfur atom.

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “Syndrome X” refers to a syndrome comprising of some or all of the following diseases: 1) dyslipoproteinemia (combined hypercholesterolemia-hypertriglyceridemia, low HDL-cholesterol), 2) obesity (in particular upper body obesity), 3) impaired glucose tolerance (IGT) leading to noninsulin-dependent diabetes mellitus (NIDDM), 4) essential hypertension and (5) thrombogenic/fibrinolytic defects.

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8th ed., Mc-Graw-Hill, Int. Ed. 1992, “Biotransformation of Drugs”, p. 13.

The following abbreviations have been used:
BOC means tert-butyloxycarbonyl,
Brine means water saturated or nearly saturated with sodium chloride,
DCM means dichloromethane,
DME means 1,2-dimethoxyethane,
DMF means dimethylformamide,
DMSO means dimethyl sulphoxide,
EDC means N-(3-dimethylaminopropyl)-N′-ethylcarbodiimide or 1-Ethyl-3-(3-dimethylaminopropyl)carbodiimide hydrochloride
EDTA means ethylenediamine tetraacetic acid,
ESI means electrospray ionization,
EtOH means ethanol,
EtOAc means ethyl acetate,
HDL means High-Density Lipoprotein,
HOBT means 1-hydroxybenzotriazole hydrate,
HPLC means High Performance Liquid Chromatography,
HRESIMS means High-Resolution Electrospray Ionization Mass Spectra,
LCMS means Liquid Chromatography Mass Spectrometry,
LRESIMS means Low-Resolution Electrospray Ionization Mass Spectra,
MeCN means acetonitrile,
MeOH means methanol,
PdCl₂(dppf).DCM means [1,1′-bis(diphenylphosphino)-ferrocene]dichloro-palladium(II) complex with DCM (1:1),
r.t. means room temperature,
R_{T} means retention time,
R_{TA} means retention time system A,
R_{TB} means retention time system B,
TBTU means N,N,N′,N′-tetramethyl-O-(benzotriazol-1-yl)uronium tetrafluoroborate,
t-BuOK means potassium tert-butoxide,
TEA means triethylamine,
TFA means trifluoroacetic acid,
THF means tetrahydrofuran.

The term “leaving group” refers to a group to be displaced from a molecule during a nucleophilic displacement reaction. Examples of leaving groups are iodide, bromide, chloride, methanesulfonyloxy, hydroxy, methoxy, thiomethoxy, toluenesulfonyloxy (tosyl) and trifluoromethanesulfonyloxy (triflate), or suitable protonated forms thereof (e.g., H₂O, MeOH).

The term “coupling agent” refers to a substance capable of catalyzing a coupling reaction, such as amidation, or esterification. Examples of coupling agents include, but are not limited to, carbonyldiimidazole, dicyclohexylcarbodiimide, pyridine, 4-dimethylamino-pyridine, and triphenylphosphine. Another example of a coupling agent is 1-ethyl-3-(3-dimethylaminopropyl)carbodiimide hydrochloride, which is used in the presence of 1-hydroxybenzotriazole and a base such as triethylamine.

The terms “exo” and “endo” are stereochemical prefixes that describe the relative configuration of a substituent on a bridge (not a bridgehead) of a bicyclic system such as 1-azabicyclo[2.2.1]heptane and bicyclo[2.2.1]heptane. If a substituent is oriented toward the larger of the other bridges, it is endo. If a substituent is oriented toward the smaller bridge it is exo. Both exo and endo forms and their mixtures are part of the present invention.

In a first aspect, the present invention provides a compound of Formula (Ia), 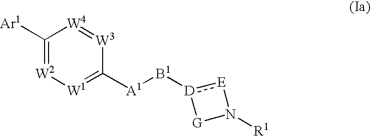
and pharmaceutically acceptable salts, hydrates, geometrical isomers, racemates, tautomers, optical isomers and N-oxides thereof; wherein:
W¹ and W³ are N and W² and W⁴ are CR¹², or W¹ and W³ are CR¹² and W² and W⁴ are N;
A¹ is CH₂, O, NR¹⁰, S, S(O) or S(O)₂;
B¹ is CH₂, O, NR¹⁰, S, S(O), S(O)₂, C(O) or CONR¹⁰, provided that when B¹ is O, NR¹⁰, S, S(O), S(O)₂, C(O) or CONR¹⁰, then A¹ is CH₂;
D is N, C or CR¹¹, provided that D must be CR¹¹ and said R¹¹ must be hydrogen or methyl when B¹ is selected from O, NR¹⁰, S, S(O), S(O)₂, and CONR¹⁰; [symbol]
is a single bond when D is N or CR¹¹ or a double bond when D is C;
E and G are independently C₁₋₃-alkylene, each optionally independently substituted with a substituent selected from the group consisting of C₁₋₃-alkyl, C₁₋₄-alkoxy, carboxy, fluoro-C₁₋₃-alkyl, hydroxy, hydroxymethyl, and fluoro, provided that the ring formed by D, E, N and G has not more than 7 ring atoms, and further provided that the said ring has 6 or 7 ring atoms when D is N, and yet further provided that the total number of substituents on E and G independently is not more than 2;
R¹ is C(O)OR², C(O)R², S(O)₂R², C(O)NR²R³, —CH₂—C(O)NR²R³, or a 5- or 6-membered heteroaryl group linked via a ring carbon atom, wherein the said heteroaryl group is optionally substituted with C₁₋₄-alkyl.

Ar¹ is phenyl which is optionally substituted in one or more positions with a substituent independently selected from:
(a) CF₃SO₃,
(b) halogen selected from chlorine, bromine and fluorine,
(c) C₁₋₄-alkylsulfinyl,
(d) —S(O)₂R⁴,
(e) —S(O)₂NR¹R⁵,
(f) —NR⁶S(O)₂R⁴,
(g) —CH₂—NR^{6C}(O)R⁴,
(h) —NR^{6C}(O)R⁴,
(i) —C(O)NR⁵R⁵,
j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) CH₃OC(O)NH—,
(p) C-heterocyclyl, optionally substituted with C₁₋₄-alkyl,
(q) —CN,
(r) —OR⁸,
(s) —SCF₃,
(t) —NO₂,
(u) phosphonooxy,
(v) C-heterocyclylsulfonyl, optionally substituted with C₁₋₄-alkyl,
(w) —NR⁵R⁵,
(x) —C(OH)CH₃CF₃,
(y) [C(OH)CH₃CF₃]—C₁₋₆-alkyl,
(z) cyano-C₁₋₆-alkyl,
(aa) guanidino,
(bb) amidino,
(cc) C₁₋₆-alkyl,
(dd) C₁₋₄-alkoxy-C₁₋₄-alkyl,
(ee) fluoro-C₁₋₄-alkyl,
(ff) C₂₋₆-alkenyl,
(gg) fluoro-C₂₋₄-alkenyl,
(hh) hydroxy-C₁₋₆-alkyl,
(ii) C₁₋₄-alkylsulfonyl-C₁₋₄-alkyl,
(jj) hydroxy-C₂₋₄-alkoxy-C₁₋₄-alkyl,
(kk) C₂₋₃-acyl-C₁₋₃-alkyl,
(ll) C₂₋₆-alkynyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with C₁₋₄-alkyl,
(qq) C₃₋₆-cycloalkyl,
(rr) C₃₋₆-cycloalkyl-C₁₋₄-alkyl,
(ss) R⁵R⁵N—C₁₋₂-alkyl,
(tt) —C(O)OR⁷,
(uu) aryl,
(w) aryl-C₁₋₄-alkyl,
(ww) aryl-C₂₋₄-alkenyl,
(xx) aryl-C₂₋₄-alkynyl,
(yy) heteroaryl,
(zz) heteroaryl-C₁₋₄-alkyl,
(aaa) heteroaryl-C₂₋₄-alkenyl, and
(bbb) heteroaryl-C₂₋₄-alkynyl,
   wherein any aryl or heteroaryl residue, alone or as part of another group, as substituent on Ar¹ is optionally substituted in one or more positions with a substituent independently selected from the group Z¹ consisting of:
(a) halogen selected from chlorine and fluorine,
(b) C₁₋₄-alkyl,
(c) hydroxy,
(d) C₁₋₄-alkoxy,
(e) —OCF₃,
(f) —SCF₃,
(g) —CN,
(h) —C(OH)CH₃CF₃,
(i) hydroxy-C₁₋₄-alkyl,
(j) —CF₃,
(k) —S(O)₂CH₃,
(l) —S(O)₂NH₂,
(m) —S(O)₂NHCH₃,
(n) —S(O)₂N(CH₃)₂,
(o) —N(CH₃)S(O)₂CH₃,
(p) —N(CH₃)C(O)CH₃,
(q) —C(O)NH₂,
(r) —C(O)NHCH₃,
(s) —C(O)N(CH₃)₂,
(t) —C(O)CH₃,
(u) —NH₂,
(v) —NHCH₃,
(w) —N(CH₃)₂,
(x) —NO₂, and
(y) methoxycarbonyl;
   R² is selected from:
(a) C₁₋₆-alkyl,
(b) C₁₋₆-alkoxy-C₂₋₆-alkyl,
(c) hydroxy-C₂₋₆-alkyl,
(d) fluoro-C₂₋₆-alkyl,
(e) C₃₋₆-alkynyl,
(f) C₃₋₆-alkenyl,
(g) C₃₋₇-cycloalkyl,
(h) C₅₋₈-cycloalkenyl,
(i) NR⁹R⁹, provided that R¹ is not selected from C(O)OR², C(O)NR²R³ and —CH₂—C(O)NR²R³,
(j) C-heterocyclyl, optionally substituted with C₁₋₄-alkyl,
(k) C₇₋₈-bicyclyl, optionally substituted with hydroxy,
(l) C₇₋₈-bicyclylmethyl,
(m) azabicyclyl, optionally substituted with hydroxy,
(n) C₃₋₇-cycloalkyl-C₄-alkyl, wherein cycloalkyl is optionally substituted with methyl,
(o) C₁₋₆-alkylsulfonyl-C₂₋₆-alkyl,
(p) C₂₋₃-acyl-C₁₋₄-alkyl,
(q) arylcarbonyl-C₁₋₄-alkyl,
(r) heteroarylcarbonyl-C₁₋₄-alkyl,
(s) [C(OH)CH₃CF₃]—C₁₋₆-alkyl,
(t) N-heterocyclylcarbonyl-C₂₋₄-alkyl, wherein heterocyclyl is optionally substituted with methyl,
(u) C-heterocyclylcarbonyl-C₂₋₄-alkyl, wherein heterocyclyl is optionally substituted with methyl,
(v) aminocarbonyl-C₂₋₆-alkyl,
(w) C₁₋₃-alkylaminocarbonyl-C₂₋₆-alkyl,
(x) di(C₁₋₃-alkyl)aminocarbonyl-C₂₋₆-alkyl,
(y) hydroxy-C₂₋₄-alkoxy-C₂₋₄-alkyl,
(z) hydroxy-C₄₋₆-cycloalkyl,
(aa) oxo-C₄₋₆-cycloalkyl,
(bb) fluoro-C₄₋₆-cycloalkyl,
(cc) C₁₋₃-alkoxy-C₄₋₆-cycloalkyl,
(dd) methyl-C₃₋₆-cycloalkyl,
(ee) oxo-N-heterocyclyl-C₂₋₄-alkyl,
(ff) fluoro-N-heterocyclyl-C₂₋₄-alkyl,
(gg) amino-N-heterocyclyl-C₂₋₄-alkyl,
(hh) hydroxy-N-heterocyclyl-C₂₋₄-alkyl,
(ii) N-heterocyclyl-C₂₋₄-alkyl, wherein heterocyclyl is optionally substituted with methyl,
(j) C-heterocyclyl-C₁₋₄-alkyl, wherein heterocyclyl is optionally substituted with methyl,
(kk) aryl,
(ll) aryl-C₁₋₄-alkyl,
(mm) aryl-C₃₋₆-alkenyl,
(nn) aryl-C₃₋₆-alkynyl,
(oo) heteroaryl,
(pp) heteroaryl-C₁₋₄-alkyl,
(qq) heteroaryl-C₃₋₆-alkenyl, and
(rr) heteroaryl-C₃₋₆-alkynyl,
   wherein any aryl or heteroaryl residue, alone or as part of another group, is optionally independently substituted in one or more position with a substituent selected from the group Z¹ as defined above;
   R³ is selected from:
(a) hydrogen,
(b) C₁₋₆-alkyl,
(c) fluoro-C₂₋₆-alkyl,
(d) hydroxy-C₂₋₆-alkyl,
(e) C₁₋₆-alkoxy-C₂₋₆-alkyl,
(f) amino-C₂₋₆-alkyl,
(g) C₁₋₃-alkylamino-C₂₋₆-alkyl,
(h) di(C₁₋₃-alkyl)amino-C₂₋₆-alkyl,
(i) cyano-C₁₋₆-alkyl, and
(j) C₁₋₆-alkylsulfonyl-C₂₋₆-alkyl;
   R⁴ is independently selected from:
(a) C₁₋₆-alkyl,
(b) fluoro-C₁₋₆-alkyl,
(c) hydroxy-C₂₋₆-alkyl,
(d) C₁₋₄-alkoxy-C₂₋₄-alkyl,
(e) C₂₋₄-acyl-C₁₋₄-alkyl,
(f) carboxy-C₁₋₃-alkyl,
(g) C₃₋₆-cycloalkyl,
(h) oxo-C₄₋₆-cycloalkyl,
(i) hydroxy-C₄₋₆-cycloalkyl,
j) fluoro-C₄₋₆-cycloalkyl,
(k) methyl-C₃₋₆-cycloalkyl,
(l) N-heterocyclylcarbonyl-C₂₋₄-alkyl, wherein heterocyclyl is optionally substituted with methyl,
(m) oxo-N-heterocyclyl-C₂₋₄-alkyl,
(n) fluoro-N-heterocyclyl-C₂₋₄-alkyl,
(o) hydroxy-N-heterocyclyl-C₂₋₄-alkyl,
(p) amino-N-heterocyclyl-C₂₋₄-alkyl,
(q) aminocarbonyl-C₂₋₄-alkyl,
(r) C₁₋₃-alkylaminocarbonyl-C₂₋₄-alkyl,
(s) di(C₁₋₃-alkyl)aminocarbonyl-C₂₋₄-alkyl,
(t) C₂₋₃-acylamino-C₂₋₄-alkyl,
(u) hydroxy-C₂₋₄-alkoxy-C₂₋₄-alkyl,
(v) C-heterocyclylcarbonyl-C₂₋₄-alkyl, wherein heterocyclyl is optionally substituted with methyl,
(w) C₃₋₆-cycloalkyl-C₁₋₂-alkyl,
(x) aryl,
(y) aryl-C₁₋₂-alkyl,
(z) heteroaryl, and
(aa) heteroaryl-C₁₋₂-alkyl,
   wherein any aryl or heteroaryl residue, alone or as part of another group, is optionally substituted in one or more positions with a substituent independently selected from the group Z² consisting of:
(a) halogen selected from chlorine and fluorine,
(b) C₁₋₄-alkoxy,
(c) hydroxymethyl,
(d) —CN,
(e) —CF₃,
(f) C₁₋₄-alkyl,
(g) —OCF₃, and
(h) —C(O)CH₃;
   R⁵ is each independently selected from:
(a) hydrogen,
(b) C₁₋₆-alkyl,
(c) C₃₋₄-cycloalkyl,
(d) fluoro-C₂₋₄-alkyl,
(e) amino-C₂₋₆-alkyl,
(f) cyano-C₁₋₆-alkyl,
(g) hydroxy-C₂₋₆-alkyl,
(h) dihydroxy-C₂₋₆-alkyl,
(i) C₁₋₄-alkoxy-C₂₋₄-alkyl,
j) C₁₋₄-alkylamino-C₂₋₄-alkyl,
(k) di(C₁₋₄-alkyl)amino-C₂₋₄-alkyl,
(l) aminocarbonyl-C₁₋₄-alkyl,
(m) C₂₋₃-acylamino-C₂₋₄-alkyl,
(n) C₁₋₄-alkylthio-C₂₋₄-alkyl,
(o) C₂₋₄-acyl-C₁₋₄-alkyl, and
(p) C₁₋₄-alkylsulfonyl-C₁₋₄-alkyl, or
   two R⁵ groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁵ groups form a piperazine ring, the nitrogen of the piperazine ring that allows the substitution is optionally substituted with C₁₋₄-alkyl;
   R⁶ is independently selected from:
(a) hydrogen,
(b) C₁₋₄-alkyl, and
(c) hydroxy-C₂₋₄-alkyl;
   R⁷ is independently selected from:
(a) hydrogen, and
(b) C₁₋₄-alkyl;
   R⁸ is independently selected from:
(a) hydrogen,
(b) C₁₋₆-alkyl,
(c) fluoro-C₁₋₆-alkyl,
(d) hydroxy-C₂₋₆-alkyl,
(e) amino-C₂₋₆-alkyl,
(f) C₁₋₃-alkylamino-C₂₋₄-alkyl,
(g) di(C₁₋₃-dialkyl)amino-C₂₋₄-alkyl,
(h) C₁₋₄-alkylsulfonyl-C₂₋₄-alkyl,
(i) N-heterocyclyl-C₂₋₄-alkyl, wherein heterocyclyl is optionally substituted with methyl,
(j) C-heterocyclyl, optionally substituted with methyl,
(k) C₂₋₃-acylamino-C₂₋₄-alkyl,
(l) [C(OH)CH₃CF₃]—C₁₋₆-alkyl,
(m) C₃₋₆-cycloalkyl,
(n) methyl-C₃₋₆-cycloalkyl,
(o) C₃₋₆-cycloalkyl-C₁₋₂-alkyl,
(p) aryl, and
(q) heteroaryl,
   wherein any aryl or heteroaryl residue is optionally substituted in one or two positions with a substituent independently selected from the group Z² as defined above;
   R⁹ is each independently selected from:
(a) C₁₋₄-alkoxy-C₂₋₄-alkyl,
(b) amino-C₂₋₄-alkyl,
(c) C₁₋₄-alkylamino-C₂₋₄-alkyl,
(d) di(C₁₋₄-alkyl)amino-C₂₋₄-alkyl,
(e) C₂₋₃-acylamino-C₂₋₄-alkyl,
(f) C₁₋₄-alkylthio-C₂₋₄-alkyl, and
(g) C₂₋₄-acyl-C₁₋₄-alkyl,
   or two R⁹ groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁹ groups form a piperazine ring, the nitrogen of the piperazine ring that allows the substitution is optionally substituted with C₁₋₄-alkyl;
   R¹⁰ is independently selected from:
(a) hydrogen,
(b) C₁₋₆-alkyl,
(c) cyclopropyl,
(d) cyclobutyl,
(e) cyclopropylmethyl,
(f) fluoro-C₂₋₆-alkyl,
(g) hydroxy-C₂₋₆-alkyl,
(h) C₁₋₂-alkoxy-C₂₋₆-alkyl,
(i) amino-C₂₋₆-alkyl,
(j) di(C₁₋₃-alkyl)amino-C₂₋₆-alkyl,
(k) C₁₋₃-alkylamino-C₂₋₆-alkyl,
(l) cyano-C₁₋₄-alkyl,
(m) C₂₋₆-acyl,
(n) C₂₋₆-acyl-C₁₋₆-alkyl,
(o) C₁₋₆-alkylsulfonyl-C₁₋₆-alkyl, and
(p) tetrahydrofuran-2-ylmethyl;
   R¹¹ is selected from:
(a) hydrogen,
(b) hydroxy,
(c) fluorine,
(d) C₁₋₄-alkoxy, and
(e) methyl;
   R¹² is each independently selected from:
(a) hydrogen,
(b) halogen selected from chlorine and fluorine,
(c) —S(O)₂CH₃,
(d) —S(O)₂CF₃,
(e) —OS(O)₂CF₃,
(f) —S(O)NH₂,
(g) —S(O)₂NHCH₃,
(h) —S(O)₂N(CH₃)₂,
(i) —NHS(O)₂CH₃,
(j) —N(CH₃)S(O)₂CH₃,
(k) —NHC(O)CH₃,
(l) —N(CH₃)C(O)CH₃,
(m) —C(O)NH₂,
(n) —C(O)NHCH₃,
(o) —C(O)N(CH₃)₂,
(p) —CN,
(q) —CF₃,
(r) guanidino,
(s) amidino,
(t) —OH,
(u) C₁₋₄-alkoxy,
(v) —OCF₃,
(w) C₃₋₅-cycloalkyloxy,
(x) —SCF₃,
(y) —NO₂,
(z) —NR⁵R⁵, wherein each R⁵ is independently selected from the group consisting of hydrogen and C₁₋₄-alkyl; or two R⁵ groups together with the nitrogen to which they are attached form a pyrrolidine or an azetidine ring,
(aa) —C(OH)CH₃CF₃,
(bb) C₁₋₃-alkyl,
(cc) C₁₋₃-alkoxy-C₁₋₂-alkyl,
(dd) C₂₋₃-acyl,
(ee) C₂₋₃-alkenyl,
(ff) hydroxy-C₁₋₄-alkyl,
(gg) fluoro-C₂₋₃-alkyl,
(hh) C₂₋₃-alkynyl, and
(ii) C₃₋₅-cycloalkyl.

A preferred group of compounds of the invention are compounds of Formula (Ib): 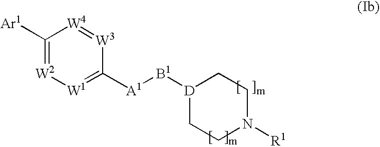
and pharmaceutically acceptable salts, hydrates, geometrical isomers, racemates, tautomers, optical isomers and N-oxides thereof; wherein:
W¹ and W³ are N and W² and W⁴ are CR¹², or W¹ and W³ are CR¹² and W² and W⁴ are N;
A¹ is CH₂, O, NR¹⁰, S, S(O) or S(O)₂;
B¹ is CH₂, O, NR¹⁰, S, S(O), S(O)₂, C(O) or CONR¹⁰, provided that when B¹ is O, NR¹⁰, S, S(O), S(O)₂, C(O) or CONR¹⁰, then A¹ is CH₂;
m is each independently 0 or 1;
D is N or CR¹¹, provided that D must be CR¹¹ and said R¹¹ must be hydrogen or methyl when B¹ is selected from O, NR¹⁰, S, S(O), S(O)₂, and CONR¹⁰, and further provided that each m is 1 when D is N;
Ar¹, Z¹, Z², R¹ to R⁹ and R¹² are as defined in Formula (Ia);
R¹⁰ is independently selected from:
(a) hydrogen,
(b) C₁₋₄-alkyl,
(c) cyclopropyl,
(d) cyclobutyl,
(e) cyclopropylmethyl,
(f) fluoro-C₂₋₄-alkyl,
(g) C₁₋₂-alkoxy-C₂₋₃-alkyl,
(h) hydroxy-C₂₋₄-alkyl,
(i) C₂₋₃-acyl,
j) amino-C₂₋₄-alkyl,
(k) methylamino-C₂₋₄-alkyl,
(l) dimethylamino-C₂₋₄-alkyl,
(m) cyano-C₁₋₄-alkyl, and
(n) tetrahydrofuran-2-ylmethyl;
   R¹¹ is selected from:
(a) hydrogen,
(b) hydroxy,
(c) fluorine, and
(d) methyl.

A further preferred group of compounds of the invention are compounds of Formula (Ic): 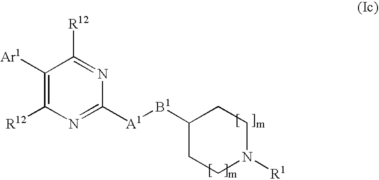
and pharmaceutically acceptable salts, hydrates, geometrical isomers, racemates, tautomers, optical isomers and N-oxides thereof; wherein:
A¹ is CH₂,O or NR¹⁰;
B¹ is CH₂, O or NR¹⁰, provided that when B¹ is O or NR¹⁰, then A¹ is CH₂;
m is each independently 0 or 1;
Z¹, Z², R¹ to R⁷, R⁹ and R¹² are as defined in Formula (Ia), provided that at least one of R¹² is hydrogen;
R¹⁰ is as defined in Formula (Ib);
Ar¹ is phenyl, which is optionally substituted in one, two or three positions with a substituent independently selected from the group Z³ consisting of:
(a) CF₃SO₃,
(b) halogen selected from bromine, chlorine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —NR^{6C}(O)R⁴,
(h) —CH₂—NR^{6C}(O)R⁴
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) —NHC(O)OCH₃,
(p) C-heterocyclyl, optionally substituted with methyl,
(q) —CN,
(r) —OR⁸,
(s) —SCF₃,
(t) —NO₂,
(u) phosphonooxy,
(v) C-heterocyclylsulfonyl, optionally substituted with methyl,
(w) —NR⁵R⁵,
(x) —C(OH)CH₃CF₃,
(y) cyano-C₁₋₆-alkyl,
(z) guanidino,
(aa) amidino,
(bb) C₁₋₆-alkyl,
(cc) C₁₋₄-alkoxy-C₁₋₄-alkyl,
(dd) fluoro-C₁₋₄-alkyl,
(ee) C₂₋₆-alkenyl,
(ff) fluoro-C₂₋₄-alkenyl,
(gg) hydroxy-C₁₋₆-alkyl,
(hh) C₁₋₄-alkylsulfonyl-C₁₋₄-alkyl,
(ii) hydroxy-C₂₋₄-alkoxy-C₁₋₄-alkyl,
(j) C₂₋₃-acyl-C₁₋₃-alkyl,
(kk) C₂₋₆-alkynyl,
(ll) C₃₋₆-cycloalkyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with methyl,
(qq) C₃₋₆-cycloalkyl-C₁₋₄-alkyl,
(rr) R⁵R⁵N—C₁₋₂-alkyl,
(ss) —C(O)OR⁷,
(tt) aryl, and
(uu) heteroaryl,
   wherein any aryl or heteroaryl residue as substituent on Ar¹ is optionally substituted in one or more positions with a substituent independently selected from the group Z¹ as defined herein for Formula (Ia);
   R⁸ is independently selected from:
(a) hydrogen,
(b) C₁₋₄-alkyl,
(c) CF₃,
(d) C₃₋₅-cycloalkyl,
(e) methyl-C₃₋₅-cycloalkyl, and
(f) C-heterocyclyl, optionally substituted with methyl.

A preferred subgroup of compounds of the general Formula (Ic) consists of compounds wherein:

A¹ is CH₂ and B¹ is O or NR¹⁰, or

A¹ is O or NR¹⁰ and B¹ is CH₂.

More preferably A¹ is O or NR¹⁰ and B¹ is CH₂;

m is each 1;

Ar¹ is phenyl, which is optionally substituted in one, two or three positions with a substituent independently selected from the group Z⁴ consisting of:

(a) halogen selected from chlorine and fluorine,
(b) C₁₋₄-alkylsulfonyl,
(c) C₁₋₄-alkylsulfinyl,
(d) hydroxy-C₂₋₄-alkylsulfonyl,
(e) C₃₋₅-cycloalkylsulfonyl,
(f) methyl-C₃₋₅-cycloalkylsulfonyl,
(g) trifluoromethylsulfonyl,
(h) —S(O)₂NR^{5A}R^{5A},
(i) C₁₋₄-alkylsulfonamido,
(j) C₂₋₄-acylamino,
(k) C₂₋₄-acylaminomethyl,
(l) carboxy-C₁₋₃-alkylcarbonylamino,
(m) —C(O)NR^{5A}R^{5A},
(n) —CH₂—C(O)NR^{5A}R^{5A}
(o) —NHC(O)OCH₃,
(p) C₂₋₄-acyl,
(q) C₃₋₅-cycloalkylcarbonyl,
(r) C₁₋₄-alkoxy,
(s) C₃₋₅-cycloalkyloxy,
(t) C-heterocyclyl,
(u) —CN,
(v) —OH,
(w) —OCF₃,
(x) —CF₃,
(y) —NO₂,
(z) —NR^{5A}R^{5A},
(aa) —C(OH)CH₃CF₃,
(bb) cyano-C₁₋₂-alkyl,
(cc) C₁₋₄-alkyl,
(dd) C₃₋₅-cycloalkyl,
(ee) C₁₋₂-alkoxy-C₁₋₂-alkyl,
(ff) vinyl,
(gg) ethynyl,
(hh) hydroxy-C₁₋₂-alkyl,
(ii) C-heterocyclyloxy, optionally substituted with methyl,
(jj) R^{5A}R^{5A} N—C₁₋₂-alkyl, and
(kk) —C(O)OR^{7A};
   R¹ is a group R^{1A} selected from C(O)OR^{2A}, C(O)R^{2A}, S(O)₂R^{2A}, C(O)NR^{2A}R^{3A}, and —CH₂—C(O)NR^{2A}R^{3A};
   R^{2A} is selected from:
(a) C₁₋₆-alkyl,
(b) C₁₋₆-alkoxy-C₂₋₆-alkyl,
(c) hydroxy-C₂₋₆-alkyl,
(d) hydroxy-C₂₋₄-alkoxy-C₂₋₄-alkyl,
(e) fluoro-C₂₋₆-alkyl,
(f) C₃₋₆-alkynyl,
(g) C₃₋₇-cycloalkyl,
(h) C₅₋₈-cycloalkenyl,
(i) NR^{9A}R^{9A} provided that R^{1A} is not selected from C(O)OR^{2A}, C(O)NR^{2A}R^{3A} and —CH₂—C(O)NR^{2A}R^{3A},
(j) C-heterocyclyl, optionally substituted with methyl,
(k) C₇₋₈-bicyclyl,
(l) 2-norbornylmethyl,
(m) azabicyclyl,
(n) C₃₋₆-cycloalkyl-C₁₋₄-alkyl, wherein cycloalkyl is optionally substituted with methyl,
(o) C₂₋₃-acyl-C₁₋₄-alkyl,
(p) arylcarbonyl-C₁₋₄-alkyl,
(q) heteroarylcarbonyl-C₁₋₄-alkyl,
(r) [C(OH)CH₃CF₃]—C₁₋₆-alkyl,
(s) N-heterocyclylcarbonyl-C₂₋₄-alkyl, wherein heterocyclyl is optionally substituted with methyl,
(t) hydroxy-C₄₋₆-cycloalkyl,
(u) oxo-C₄₋₆-cycloalkyl,
(v) fluoro-C₄₋₆-cycloalkyl,
(w) methoxy-C₄₋₆-cycloalkyl,
(x) methyl-C₃₋₆-cycloalkyl,
(y) oxo-N-heterocyclyl-C₂₋₄-alkyl,
(z) hydroxy-N-heterocyclyl-C₂₋₄-alkyl,
(aa) fluoro-N-heterocyclyl-C₂₋₄-alkyl,
(bb) amino-N-heterocyclyl-C₂₋₄-alkyl,
(cc) N-heterocyclyl-C₂₋₄-alkyl, wherein heterocyclyl is optionally substituted with methyl,
(dd) C-heterocyclyl-C₁₋₄-alkyl, wherein heterocyclyl is optionally substituted with methyl,
(ee) aryl,
(ff) aryl-C₁₋₄-alkyl,
(gg) heteroaryl, and
(hh) heteroaryl-C₁₋₄-alkyl,
   wherein any aryl or heteroaryl residue, alone or as a part of another group, is optionally substituted in one or more positions with a substituent independently selected from the group Z⁵ consisting of:
(a) halogen selected from chlorine and fluorine,
(b) methyl,
(c) ethyl,
(d) methoxy,
(e) ethoxy,
(f) isopropoxy,
(g) hydroxy,
(h) —OCF₃,
(i) —CF₃,
(i) —CN,
(k) —C(OH)CH₃CF₃,
(l) dimethylamino,
(m) hydroxymethyl,
(n) —S(O)₂CH₃,
(o) —C(O)CH₃, and
(p) —C(O)NH₂;
   R^{3A} is selected from:
(a) hydrogen,
(b) C₁₋₄-alkyl,
(c) hydroxy-C₂₋₄-alkyl, and
(d) methoxy-C₂₋₄-alkyl;
   R^{5A} is each independently selected from:
(a) hydrogen,
(b) C₁₋₃-alkyl,
(c) C₁₋₂-alkoxy-C₂₋₄-alkyl,
(d) C₃₋₄-cycloalkyl,
(e) hydroxy-C₂₋₄-alkyl,
(f) cyano-C₁₋₃-alkyl,
(g) C₂₋₃-acylamino-C₂₋₃-alkyl,
(h) dihydroxy-C₂₋₄-alkyl,
(i) aminocarbonyl-C₁₋₂-alkyl, and
(j) di(C₁₋₂-alkyl)amino-C₂₋₃-alkyl,
   or two R^{5A} groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R^{5A} groups form a piperazine ring, the nitrogen of the piperazine ring that allows the substitution is optionally substituted with methyl;
   R^{7A} is independently from:
(a) hydrogen, and
(b) C₁₋₄-alkyl;
   Two groups R^{9A} together with the nitrogen to which they are attached form a heterocyclic ring, wherein said heterocyclic ring may be optionally substituted with: i) one hydroxy or amino group, ii) one or two fluorine atoms, or iii) one or two oxo groups, provided that when the substituent is selected from fluorine, hydroxy and amino, said substituent is attached to the heterocyclic ring at a position other than alpha to a heteroatom; and when the two R^{9A} groups form a piperazine ring, the nitrogen of the piperazine ring that allows the substitution is optionally substituted with methyl;
   R¹⁰ is independently selected from:
(a) hydrogen, and
(b) C₁₋₃-alkyl;
   R¹² is each hydrogen.

A yet more preferred subgroup of compounds of Formula (Ic) consists of compounds wherein A¹ is CH₂ and B¹ is O or NR¹⁰, or A¹ is O or NR¹⁰ and B¹ is CH₂.

More preferably, A¹ is O or NR¹⁰ and B¹ is CH₂.

In another more preferred subgroup of compounds of Formula (Ic), Ar¹ is selected from methylsulfonylphenyl, [(methoxycarbonyl)amino]phenyl, (dimethylamino)carbonylphenyl, (acetylamino)phenyl, [(diethylamino)carbonyl]phenyl, (aminocarbonyl)phenyl, [(methyl-sulfonyl)amino]phenyl, (morpholin-4-ylcarbonyl)phenyl, (aminosulfonyl)phenyl, [(2-hydroxyethyl)sulfonyl]phenyl, (morpholin-4-ylsulfonyl)phenyl, [(2,5-dioxoimidazolidin-1-yl)methyl]phenyl, [(dimethylamino)sulfonyl]phenyl, {[2-(dimethylamino)ethyl]amino-carbonyl}phenyl, {[(2-hydroxymethyl)pyrrolidin-1-yl]carbonyl}phenyl, [(2,5-dioxo-pyrrolidin-1-yl)methyl]phenyl, [(4-methylpiperazin-1-yl)carbonyl]phenyl, (difluoro)-hydroxyphenyl, fluoro-[(propylamino)carbonyl]phenyl, (aminocarbonyl)fluorophenyl, {[3-(dimethylamino)pyrrolidin-1-yl]carbonyl}phenyl, [(3-hydroxypyrrolidin-1-yl)carbonyl]-phenyl and (hydroxymethyl)phenyl.

More preferably, Ar¹ is selected from 4-methylsulfonylphenyl, 4-[(methoxycarbonyl)-amino]phenyl, 4-[(dimethylamino)carbonyl]phenyl, 4-(acetylamino)phenyl, 4-[(diethyl-amino)carbonyl]phenyl, 4-(aminocarbonyl)phenyl, 4-[(methylsulfonyl)amino]phenyl, 4-(morpholin-4-ylcarbonyl)phenyl, 4-(aminosulfonyl)phenyl, 4-[(2-hydroxyethyl)sulfonyl]-phenyl, 4-(morpholin-4-ylsulfonyl)phenyl, 4-[(2,5-dioxoimidazolidin-1-yl)methyl]phenyl, 4-[(dimethylamino)sulfonyl]phenyl, 4-({[2-(dimethylamino)ethyl]amino}carbonyl)phenyl, 4-{[2-(hydroxymethyl)pyrrolidin-1-yl]carbonyl}phenyl, 4-[(2,5-dioxopyrrolidin-1-yl)-methyl]phenyl, 4-[(4-methylpiperazin-1-yl)carbonyl]phenyl, 3,5-difluoro-4-hydroxy-phenyl, 4-{[3-(dimethylamino)pyrrolidin-1-yl]carbonyl}phenyl, 3-fluoro-4-[(propyl-amino)carbonyl]phenyl, 4-(aminocarbonyl)-3-fluorophenyl, 4-[(3-hydroxypyrrolidin-1-yl)-carbonyl]phenyl and 4-(hydroxymethyl)phenyl.

In another more preferred subgroup of compounds of Formula (Ic), R^{1A} is selected from C(O)OR^{2A} and C(O)R^{2A}.

In one embodiment, R^{1A} is C(O)OR^{1A}, wherein R^{2A} is selected from C₁₋₆-alkyl and benzyl. Preferably, R^{2A} is selected from tert-butyl, benzyl, isopropyl and ethyl.

In another embodiment, R^{1A} is C(O)R^{2A}, wherein R^{2A} is selected from C₁₋₆-alkyl and phenyl. Preferably, R^{2A} is selected from 2,2-dimethylpropyl and phenyl;

In another more preferred subgroup of compounds of formula (Ic), R¹⁰ is independently selected from hydrogen and methyl. More preferably, R¹⁰ is hydrogen.

Particulary preferred compounds of Formula (Ic) are the compounds selected from the group consisting of:
tert-Butyl 4-[({5-[4-(hydroxymethyl)phenyl]pyrimidin-2-yl}oxy)methyl]piperidine-1-carboxylate;
tert-Butyl 4-[({5-[4-(methylsulfonyl)phenyl]pyrimidin-2-yl}oxy)methyl]piperidine-1-carboxylate;
Benzyl 4-[({5-[4-(methylsulfonyl)phenyl]pyrimidin-2-yl}oxy)methyl]piperidine-1-carboxylate;
   2-[(1-Benzoylpiperidin-4-yl)methoxy]-5-[4-(methylsulfonyl)phenyl]pyrimidine;
tert-Butyl 4-{[(5-{4-[(methoxycarbonyl)amino]phenyl}pyrimidin-2-yl)oxy]methyl}-piperidine-1-carboxylate;
tert-Butyl 4-{[(5-{4-[(dimethylamino)carbonyl]phenyl}pyrimidin-2-yl)oxy]methyl}-piperidine-1-carboxylate;
tert-Butyl 4-[({5-[4-(acetylamino)phenyl]pyrimidin-2-yl}oxy)methyl]piperidine-1-carboxylate;
tert-Butyl 4-{[(5-{4-[(diethylamino)carbonyl]phenyl}pyrimidin-2-yl)oxy]methyl}-piperidine-1-carboxylate;
tert-Butyl 4-[({5-[4-(aminocarbonyl)phenyl]pyrimidin-2-yl}oxy)methyl]piperidine-1-carboxylate;
tert-Butyl 4-{[(5-{4-[(methylsulfonyl)amino]phenyl}pyrimidin-2-yl)oxy]methyl}-piperidine-1-carboxylate;
tert-Butyl 4-[({5-[4-(morpholin-4-ylcarbonyl)phenyl]pyrimidin-2-yl}oxy)methyl]-piperidine-1-carboxylate;
tert-Butyl 4-[({5-[4-(aminosulfonyl)phenyl]pyrimidin-2-yl}oxy)methyl]piperidine-1-carboxylate;
tert-Butyl 4-{[(5-{4-[(2-hydroxyethyl)sulfonyl]phenyl}pyrimidin-2-yl)oxy]methyl}-piperidine-1-carboxylate;
tert-Butyl 4-[({5-[4-(morpholin-4-ylsulfonyl)phenyl]pyrimidin-2-yl}oxy)methyl]-piperidine-1-carboxylate;
tert-Butyl 4-{[(5-{4-[(2,5-dioxoimidazolidin-1-yl)methyl]phenyl}pyrimidin-2-yl)-oxy]methyl}piperidine-1-carboxylate;
tert-Butyl 4-{[(5-{4-[(dimethylamino)sulfonyl]phenyl}pyrimidin-2-yl)oxy]methyl}-piperidine-1-carboxylate;
tert-Butyl 4-[({5-[4-({[2-(dimethylamino)ethyl]amino}carbonyl)phenyl]pyrimidin-2-yl}oxy)methyl]piperidine-1-carboxylate;
tert-Butyl 4-[({5-[4-(aminocarbonyl)-3-fluorophenyl]pyrimidin-2-yl}oxy)methyl]-piperidine-1-carboxylate;
Isopropyl 4-[({5-[4-(methylsulfonyl)phenyl]pyrimidin-2-yl}amino)methyl]-piperidine-1-carboxylate;
Ethyl 4-[({5-[4-(methylsulfonyl)phenyl]pyrimidin-2-yl}amino)methyl]piperidine-1-carboxylate;
N-{[1-(3,3-dimethylbutanoyl)piperidin-4-yl]methyl}-5-[4-(methylsulfonyl)phenyl]-pyrimidin-2-amine;
tert-Butyl 4-[({5-[4-(methylsulfonyl)phenyl]pyrimidin-2-yl}amino)methyl]-piperidine-1-carboxylate;
Benzyl 4-[({5-[4-(methylsulfonyl)phenyl]pyrimidin-2-yl}amino)methyl]piperidine-1-carboxylate;
tert-Butyl 4-({[5-(4-{[(2R)-2-(hydroxymethyl)pyrrolidin-1-yl]carbonyl}phenyl)-pyrimidin-2-yl]oxy}methyl)piperidine-1-carboxylate;
tert-Butyl 4-{[(5-{4-[(2,5-dioxopyrrolidin-1-yl)methyl]phenyl}pyrimidin-2-yl)oxy]-methyl}piperidine-1-carboxylate;
tert-Butyl 4-{[(5-{4-[(4-methylpiperazin-1-yl)carbonyl]phenyl}pyrimidin-2-yl)oxy]-methyl}piperidine-1-carboxylate;
tert-Butyl 4-({[5-(3,5-difluoro-4-hydroxyphenyl)pyrimidin-2-yl]oxy}methyl)-piperidine-1-carboxylate;
tert-Butyl 4-({[5-(4-{[3-(dimethylamino)pyrrolidin-1-yl]carbonyl}phenyl)pyrimidin-2-yl]oxy}methyl)piperidine-1-carboxylate;
tert-Butyl 4-{[(5-{3-fluoro-4-[(propylamino)carbonyl]phenyl}pyrimidin-2-yl)oxy]-methyl}piperidine-1-carboxylate; and
tert-Butyl 4-{[(5-{4-[(3-hydroxypyrrolidin-1-yl)carbonyl]phenyl}pyrimidin-2-yl)-oxy]methyl}piperidine-1-carboxylate.

A further preferred group of compounds of the invention are those of Formula (Id): 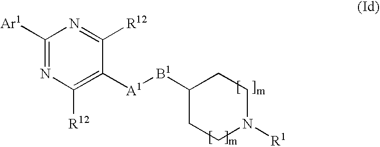
and pharmaceutically acceptable salts, hydrates, geometrical isomers, racemates, tautomers, optical isomers and N-oxides thereof; wherein:
A¹ is CH₂, O or NR¹⁰;
B¹ is CH₂, O or NR¹⁰, provided that when B¹ is O or NR¹⁰, then A¹ is CH₂;
m is each independently 0 or 1;
Z¹, Z², R¹ to R⁷, R⁹ and R¹² are as defined in Formula (Ia), provided that at least one of R¹² is hydrogen;
R⁸ is as defined in Formula (Ic);
R¹⁰ is as defined in Formula (Ib);
Ar¹ is phenyl which is optionally substituted in one or two positions with a substituent independently selected from the group Z³ as defined in Formula (Ic).

A preferred subgroup of compounds of the general Formula (Id) consists of compounds wherein:

A¹ is CH₂ and B¹ is O or NR¹⁰, or

A¹ is O or NR¹⁰ and B¹ is CH₂;

m is each 1;

Ar¹ is phenyl, which is optionally substituted in one or two positions with a substituent independently selected from the group Z⁴ as defined in Formula (Ic);

Z⁵ is as defined in Formula (Ic);

R¹ is a group R^{1A}, wherein R^{1A} is as defined in Formula (Ic);

R^{2A}, R^{3A}, R^{5A}, R^{7A} and R^{9A} are as defined in Formula (Ic);

R¹⁰ is independently selected from:

(a) hydrogen, and
(b) C₁₋₃-alkyl;
   R¹² is each hydrogen.

In a more preferred subgroup of compounds of Formula (Id), Ar¹ is CH₂ and B′ is NR¹⁰.

In another more preferred subgroup of compounds of Formula (Id), Ar¹ is C₁₋₄-alkyl-sulfonylphenyl. It is especially preferred for Ar¹ to be methylsulfonylphenyl.

In another more preferred subgroup of compounds of Formula (Id), R^{1A} is C(O)OR^{2A}. R^{2A} is preferably C₁₋₄-alkyl, more preferably tert-butyl.

In yet another more preferred subgroup of compounds of Formula (Id), R¹⁰ is selected from hydrogen, methyl and ethyl.

Particulary preferred compounds of Formula (Id) are the compounds selected from the group consisting of:
tert-Butyl 4-[({2-[4-(methylsulfonyl)phenyl]pyrimidin-5-yl}methyl)amino]-piperidine-1-carboxylate; and
tert-Butyl 4-[methyl({2-[4-(methylsulfonyl)phenyl]pyrimidin-5-yl}methyl)amino]-piperidine-1-carboxylate.

All isomeric forms possible (pure enantiomers, diastereomers, tautomers, racemic mixtures and unequal mixtures of two enantiomers) for the compounds delineated are within the scope of the invention. When the compounds described herein contain olefinic double bonds of geometric asymmetry, it is intended to include both trans and cis (E and Z) geometric isomers.

The compounds of the Formula (Ia) to (Id) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Another object of the present invention is a compound of Formula (Ia) to (Id) for use in therapy. The compound can be used in the treatment or prophylaxis of disorders relating to GPR119. Examples of such disorders are Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is a method for the treatment or prophylaxis of disorders related to GPR119, said method comprising administering to a subject (e.g., mammal, human, or animal) in need of such treatment an effective amount of a compound as described above. The GPR119-related disorder is any disorder or symptom wherein GPR119 is involved in the process or presentation of the disorder or the symptom. The GPR119-related disorders include, but are not limited to Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is a method for modulating the GPR119 receptor activity (e.g., agonizing human GPR119), comprising administering to a subject (e.g., mammal, human, or animal) in need thereof an effective amount of a compound as described above or a composition comprising a compound as described above.

Another object of the present invention is the use of a compound as described above in the manufacture of a medicament for use in the treatment or prophylaxis of Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is the use of a compound of Formula (Ia) to (Id), as described above, in the manufacture of a medicament for use in the treatment or prophylaxis of disorders related to GPR119, said method comprising administering to a subject (e.g., mammal, human, or animal) in need of such treatment an effective amount of a compound as described above. The GPR119-related disorder is any disorder or symptom wherein GPR119 is involved in the process or presentation of the disorder or the symptom. The GPR119-related disorders include, but are not limited to, Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction. Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabelling/assay techniques, blotting/chemiluminescence methods, real-time PCR, and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

The compounds of formula (Ia) to (Id) may be administered with other active compounds for the treatment of diabetes and/or obesity, for example insulin and insulin analogs, DPP-IV inhibitors, sulfonyl ureas, biguanides, α2 agonists, glitazones, PPAR-γ agonists, mixed PPAR-α/γ agonists, RXR agonists, α-glucosidase inhibitors, PTP1B inhibitors, 11-β-hydroxy steroid dehydrogenase Type 1 inhibitors, phosphodiesterase inhibitors, glycogen phosphorylase inhibitors, MCH-1 antagonists, CB-1 antagonists (or inverse agonists), amylin antagonists, CCK receptor agonists, β₃-agonists, leptin and leptin mimetics, serotonergic/dopaminergic antiobesity drugs, gastric lipase inhibitors, pancreatic lipase inhibitors, fatty acid oxidation inhibitors, lipid lowering agents and thyromimetics. It is particularly preferred that the compounds of formula (Ia) to (Id) are administered in combination with a DPP-IV inhibitor. The term “DPP-IV inhibitor” means a compound which inhibits, antagonizes or decreases the activity of dipeptidyl peptidase IV (EC 3.4.14.5). The said DPP-IV inhibitor can e.g. be a compound as disclosed in WO 2005/056003; WO 2005/056013; WO 2005/095343; WO 2005/113510; WO 2005/120494; WO 2005/121131; WO 2005/121089; WO 2006/013104; or WO 2006/076231, including references therein.

In a further aspect the invention relates to methods of making compounds of any of the formulae herein comprising reacting any one or more of the compounds of the formulae delineated herein, including any processes delineated herein. The compounds of the Formula (Ia) to (Id) above may be prepared by, or in analogy with, conventional methods. The preparation of intermediates and compounds according to the examples of the present invention may in particular be illuminated by the following Schemes 1-3. 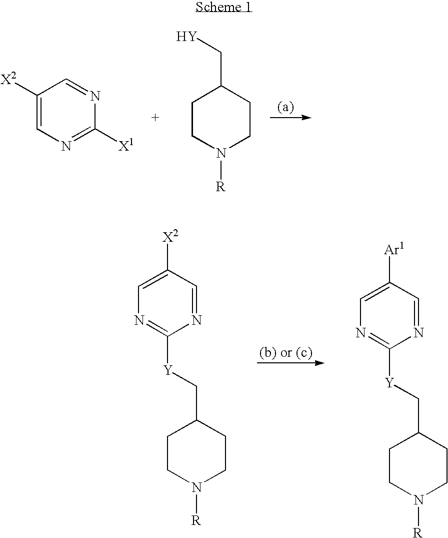
X¹ is Cl, Br;
X² is Cl, Br, I;
Y is O or NH;
R is Boc;
Ar¹ is as defined in Formula (Ia).
Reagents and Conditions:
(a) suitable base, such as NaH or t-BuOK; in a suitable solvent, such as DMF, DMSO or THF; at ambient or elevated temperature;
(b) appropriate arylboronic acid; appropriate catalyst, such as Pd(PPh₃)₄; a suitable base, such as K₂CO₃ or NaHCO₃; in a suitable solvent mixture such as 1,4-dioxane and water; at elevated temperature, for example 90° C.;
(c) (i) bis(neopentyl glycolato)diboron; suitable base, such as KOAc; appropriate catalyst, such as PdCl₂(dppf).DCM; in a suitable solvent, such as DME; at elevated temperature, for example 120° C. (microwaves); (ii) appropriate aryl halide; suitable base, such as NaHCO₃ or K₂CO₃; appropriate catalyst, such as Pd(PPh₃)₄; in a suitable solvent mixture, such as water and DME; at elevated temperature, for example 120° C. (microwaves). 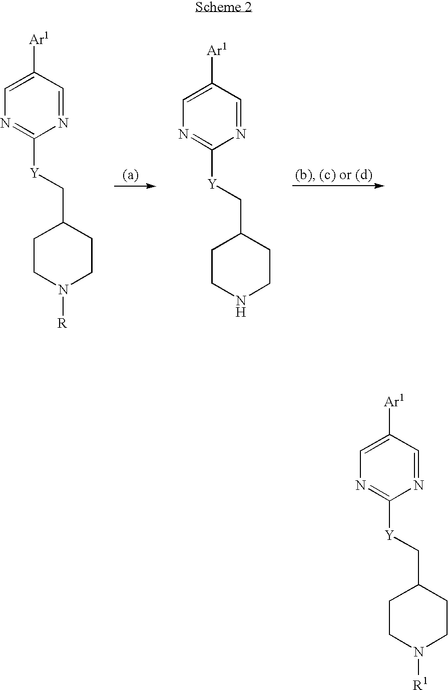
   Y is O or NH;
   Ar¹ is as defined in Formula (Ia);
   R is Boc;
   R¹ is as defined in Formula (Ia).
   Reagents and Conditions:
(a) suitable deprotecting agent, such as TFA, HCl (g) or aqueous concentrated HCl; in a suitable solvent, such as DCM or ethanol; at ambient or elevated temperature;
(b) (i) appropriate carboxylic acid; suitable base, such as triethylamine; in suitable solvent, such as THF, dioxane or DMF; (ii) appropriate coupling reagent, such as HOBT/EDC, propylphosphonic anhydride or TBTU;
(c) appropriate acid chloride or chloroformate; suitable base, such as triethylamine; in suitable solvent, such THF or DMF;
(d) appropriate alcohol; suitable coupling reagents, such as 1,1′-carbonylbis(1H-imidazole); in suitable solvent, such DCM, acetonitile or DCM/THF; at elevated temperature. 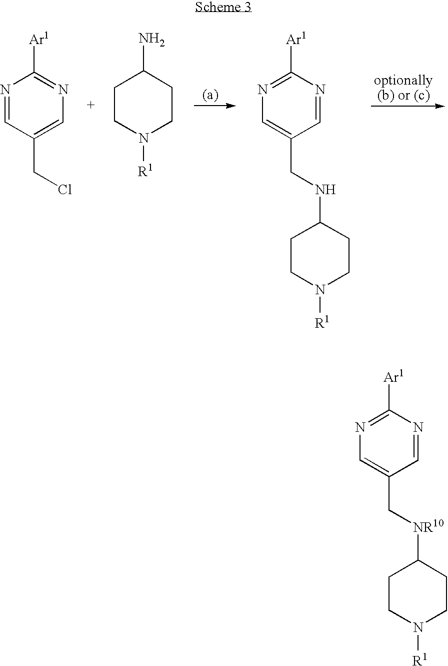
   Ar¹ is as defined in Formula (Ia);
   R¹ is Boc;
   R¹⁰ is as defined in Formula (Ia).
   Reagents and Conditions:
(a) appropriate base, such as N,N-diisopropylethylamine or triethylamine; in a suitable solvent, such as acetonitrile; at elevated temperature, for example 60° C.;
(b) appropriate aldehyde or ketone corresponding to R¹⁰; appropriate reducing agents, e.g., NaBH(OAc)₃ or NaBH₃CN; in a suitable solvent, such as MeOH, 1,2-dichloroethane, DCM, or in a solvent mixture such as methanol/water; at ambient or elevated temperature;
(c) appropriate alkylating agent corresponding to R¹⁰, such as alkylhalide, alkyltriflate; suitable base, such N,N-diisopropylethyl amine or triethylamine; in a suitable solvent, such as THF or DMF; at elevated temperature.

Definitions of variables in the structures in the schemes herein are commensurate with those of corresponding positions in the formulae delineated herein.

The processes described below in the example section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of Formula (Ia) to (Id) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of Formula (Ia) to (Id) and other compounds herein are either known or may be prepared in analogy with the preparation of known compounds.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The invention will now be further illustrated by the following non-limiting Examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

### EXAMPLES AND INTERMEDIATE COMPOUNDS

### Experimental Methods

¹H Nuclear magnetic resonance (NMR) and ¹³C NMR were recorded on a Bruker Advance DPX 400 spectrometer at 400.1 MHz and 100.6 MHz, respectively. All spectra were recorded using residual solvent or tetramethylsilane (TMS) as internal standard. Low-resolution electrospray ionization mass spectra (LRESIMS) were obtained using an Agilent MSD mass spectrometer or a Waters ZQ mass spectrometer. High-resolution electrospray ionization mass spectra (HRESIMS) were obtained on an Agilent LC/MSD TOF connected to an Agilent 1100 LC-system, Ion Source: ESI, Ion polarity: pos, Data: profile mode, Scan range: 100-1100 Da, MS parameters: Fragmentor 215V, Skimmer 560V och OCT RF (octpole rods) 250 V.; Reference Masses 121.050873 and 922.009798 (Agilent reference Mix); LC: A 15 mM ammonium acetate; B 100 MeCN; flow 400 μL/min isocratic. Flash chromatography was performed on Merck silica gel 60 (230-400 mesh). Microwave irraditions were carried out using the Smith Creator or Optimizer (Personal Chemistry) using 0.5-2 mL or 2-5 mL Smith Process vials fitted with aluminum caps and septa. The compounds were automatically named using ACD 6.0.

Analytical LCMS Data were Obtained with:

System A: Agilent MSD mass spectrometer; Agilent 1100 system; ACE 3 C8 column (50×3.0 mm); Water containing 0.1% TFA and acetonitrile were used as mobile phases at a flow rate of 1 mL/min with gradient times of 3.0 min (gradient 10-97% acetonitrile); or

System B: Agilent MSD mass spectrometer; Agilent 1100 system; YMC ODS-AQ column (33×3.0 mm); Water containing 0.1% TFA and acetonitrile were used as mobile phases at a flow rate of 1 mL/min with gradient times of 3.0 min (gradient 10-97% acetonitrile); or

System C: Waters ZQ mass spectrometer; Waters 996 PDA detector (DAD 215-395 nm); ACE C8 (3 gm) column (30×3.0 mm) (from ACT); Water containing 10 mM ammonium acetate (pH=7) and acetonitrile were used as mobile phases at a flow rate of 1 mL/min with gradient times of 3.2 min (gradient 5-100% acetonitrile).

Preparative HPLC was Performed on Gilson System Equipped with:

System D: ACE C8 5 μm (21.2×50 mm) column. Water containing 0.1% TFA and acetonitrile were used as mobile phases at a flow rate of 25 mL/min with gradient times of 6 min.; or

System E: XTerra Prep MS C18 5 μm (19×50 mm) column. Water containing 50 mM NH₄HCO₃ (pH=10) and acetonitrile were used as mobile phases at a flow rate of 25 mL/min with gradient times of 6 min; or Xterra MS C18 5 μm (30×100 mm) column. Water containing 50 mM NH₄HCO₃ (pH=10) and acetonitrile were used as mobile phases at a flow rate of 40 mL/min with gradient times of 8.5 min; or

System F: YMC ODS-AQ 10 μM (30×150 mm) column. Water containing 0.1% TFA and acetonitrile were used as mobile phases at a flow rate of 45 mL/min with gradient times of 8.5 min.

Methods for Preparation

General Method A1: Suzuki-Type Coupling Reaction.

tert-Butyl 4 {[(5-bromopyrimidin-2-yl)oxy]methyl}piperidine-1-carboxylate (Intermediate A1; 30 mg, 0.08 mmol), arylboronic acid (0.089 mmol) and NaHCO₃ (17 mg, 0.20 mmol) was mixed with 80% aqueous dioxane (0.8 mL) and [(C₆H₅)₃P]₄Pd (5 mg, 0.004 mmol). The mixture was stirred at 85° C. over 4 h. The mixture was then filtered and evaporated and purified by preparative HPLC (System D).

General Method A2: Suzuki-Type Coupling Reaction.

The same method as A1 with the exception that K₂CO₃ (28 mg, 0.20 mmol) was used instead of NaHCO₃.

General Method B: Palladium-Catalyzed Cross-Coupling Reaction (In Situ Generated arylboronic acid).

tert-Butyl 4 {[(5-bromopyrimidin-2-yl)oxy]methyl}piperidine-1-carboxylate (Intermediate A1; 74 mg, 0.2 mmol), bis(neopentylglycolato)diboron (68 mg, 0.3 mmol) and KOAc (88 mg, 0.9 mmol) were mixed with DME (2 mL). Then PdCl₂(dppf).DCM (16 mg, 0.02 mmol) was added. The mixture was heated to 100° C. over 1 h (or 85° C. over 2 h). Aryl halide (0.4 mmol), K₂CO₃ (55 mg, 0.4 mmol), [(C₆H₅)₃P]₄Pd (12 mg, 0.01 mmol), DME (1 mL) and water (0.8 mL) were added and the mixture was stirred at 85° C. over 2 h. EtOH was added and the mixture was filtered. The filtrate was concentrated under reduced pressure and EtOAc (5 mL) was added. The organic phase was separated and filtered. The solvent was evaporated and the residue was purified by preparative HPLC (System E, gradient 20-50% MeCN).

### Intermediate A1

### tert-Butyl 4 {[(5-bromopyrimidin-2-yl)oxy]methyl}piperidine-1-carboxylate

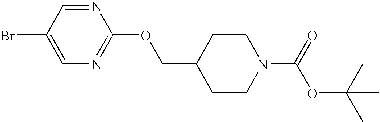

A mixture of tert-butyl 4-(hydroxymethyl)piperidine-1-carboxylate (1.32 g, 4.80 mmol) and sodium tert-butoxide (460 mg, 4.80 mmol) in THF (20 mL) was stirred for 15 min. Then 5-bromo-2-chloropyrimidine (772 mg, 4.00 mmol) was added and the mixture was heated at 60° C. overnight. The solvent was concentrated under reduced pressure and water and EtOAc were added. The organic phase was separated, concentrated, and the crude product was purified by flash-chromatography using EtOAc:toluene (1:4) as eluent. Yield 0.551 g (37%). Analytical HPLC: purity 100% (System B, R_{T}=2.49 min); ¹H NMR (400 MHz, CDCl₃) δ ppm 1.19-1.33 (m, 2H), 1.45 (s, 9H), 1.76-1.85 (m, 2H), 1.98 (m, 1H), 2.72 (m, 2H), 4.06-4.21 (m, 4H), 8.81 (s, 2H); LRESIMS for C₁₅H₂₂BrN₃O₃ m/z 272 (M-Boc)⁺.

### Intermediate A2

### 4-(2-{[1-(tert-butoxycarbonyl)piperidin-4-yl]methoxy}pyrimidin-5-yl)benzoic acid

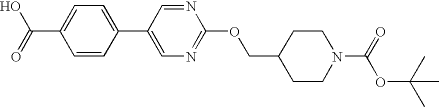

tert-Butyl 4 {[(5-bromopyrimidin-2-yl)oxy]methyl}piperidine-1-carboxylate (372 mg, 1.00 mmol; Intermediate A1) and 4-methoxycarbonylphenyl boronic acid (225 mg, 1.25 mmol) were dissolved in dioxane-water (4:1; 8 mL) and NaHCO₃ (210 mg, 2.5 mmol) was added. Then [(C₆H₅)₃P]₄Pd (59 mg, 0.05 mmol) was added and the mixture heated to 80° C. 1 h. The mixture was cooled and HOAc (0.12 mL) was added. The mixture was stirred at r.t. overnight. The solvent was evaporated and the residue partitioned between DCM and water. The organic phase was separated and washed with 5% aqueous NaHCO₃. The crude product (0.488 g) was purified by flash chromatography on silica using 2% MeOH in CHCl₃ as eluent. Yield 345 mg (81%) of tert-butyl 4-[({5-[4-(methoxycarbonyl)-phenyl]pyrimidin-2-yl}oxy)methyl]piperidine-1-carboxylate. Analytical HPLC: purity 98%, R_{T}=2.52 min (System A), HPLC 98%, R_{T}=2.45 min (System B); ¹H NMR (400 MHz, CDCl₃) δ ppm 1.21-1.37 (m, 2H), 1.45 (s, 9H), 1.81-1.90 (m, 2H), 2.04 (m, 1H), 3.94 (s, 3H), 4.10-4.20 (m, 2H), 4.28 (d, J=6.5 Hz, 2H), 7.59 (m, 2H), 8.14 (m, 2H), 8.74 (s, 2H); LRESIMS for m/z 428 (M+H)⁺.

This intermediate, tert-butyl 4-[({5-[4-(methoxycarbonyl)phenyl]pyrimidin-2-yl}oxy)-methyl]piperidine-1-carboxylate, (309 mg, 0.72 mmol) was dissolved in a mixture of MeOH (2 mL) and THF (2 mL). Then 2 M LiOH (0.72 mL) was added. The mixture was stirred at r.t. over 1 h then MeOH (4 mL) was added and the mixture was heated at 60° C. for 1 h 40 min. The mixture was neutralized with 1 M HCl (1.8 mL), extracted with DCM and concentrated to give Intermediate A2. Yield 236 mg (79%). Analytical HPLC: purity 85%, R_{T}=2.22 min (System A), min); ¹H NMR (400 MHz, CD₃OD-CDCl₃) δ ppm 1.22-1.37 (m, 2H), 1.45 (s, 9H), 1.78-1.90 (m, 2H), 2.06 (m, 1H), 2.79 (m, 2H), 4.12 (m, 2H), 4.32 (d, J=6.5 Hz, 2H), 7.70 (m, 2H), 8.13 (m, 2H), 8.83 (s, 2H); LRESIMS for C₂₂H₂₇N₃O₅ m/z 414 (M+H)⁺.

### Intermediate A3

### 5-[4-(Methylsulfonyl)phenyl]-N-(piperidin-4-ylmethyl)pyrimidin-2-amine

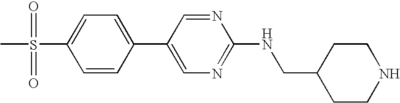

tert-Butyl 4-[({5-[4-(methylsulfonyl)phenyl]pyrimidin-2-yl}amino)methyl]piperidine-1-carboxylate (0.40 g, 0.85 mmol; obtained in Example A22) was dissolved in DCM (3.2 mL) and TFA (0.8 mL) was added. After 40 min the mixture was concentrated under reduced pressure. The residue was dissolved in DCM and washed with 2 M NaOH and dried (Na₂SO₄) to give Intermediate A3. Yield 266 mg (76%). Analytical HPLC: purity 100% (System A, R_{T}=1.11 min); ¹H NMR (400 MHz, CD₃OD) δ ppm 1.15-1.38 (m, 2H), 1.69-1.99 (m, 5H), 2.61 (m, 1H), 2.96-3.16 (m, 5H), 3.38 (m, 2H), 5.42 (m, 1H), 7.65 (m, 2H), 7.99 (m, 2H), 8.54 (s, 2H); LRESIMS for C₁₇H₂₂N₄O₂S m/z 347 (M+H)⁺.

### Example A1

### tert-butyl 4-[({5-[4-(hydroxymethyl)phenyl]pyrimidin-2-yl}oxy)methyl]piperidine-1-carboxylate

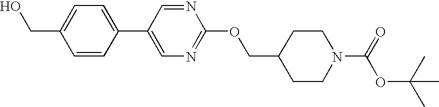

The intermediate A1 (298 mg, 0.80 mmol) and 4-(hydroxymethylphenyl)boronic acid (146 mg, 0.96 mmol) were mixed with dioxane (4 mL) and K₂CO₃ (276 mg, 2.00 mmol) dissolved in water (1 mL) was added. [(C₆H₅)₃P]₄Pd (46 mg, 0.04 mmol) was added and the mixture was stirred at 85° C. over 2 h. The mixture was cooled and filtered and the filtrate was concentrated under reduced pressure and the residue mixed with 5% aqueous NaHCO₃ and DCM. The organic phase was separated, dried (Na₂SO₄), filtered and concentrated. The residue was purified via flash chromatography using 3% MeOH in CHCl₃ as eluent to give the title compound. Yield 235 mg (73%). Analytical HPLC: purity 100% (System A, R_{T}=2.20 min); ¹H NMR (400 MHz, CDCl₃) δ ppm 1.21-1.36 (m, 2H), 1.45 (s, 9H), 1.72-1.90 (m, 3H), 2.04 (m, 1H), 2.74 (m, 2H), 4.08-4.21 (m, 2H), 4.25 (d, J=6.5 Hz, 2H), 4.75 (s, 2H), 7.44-7.53 (m, 4H), 8.69 (s, 2H); LRESIMS for C₂₂H₂₉N₃O₄ m/z 400 (M+H)⁺.

### Example A2

### tert-Butyl 4-[({5-[4-(methylsulfonyl)phenyl]pyrimidin-2-yl}oxy)methyl]piperidine-1-carboxylate

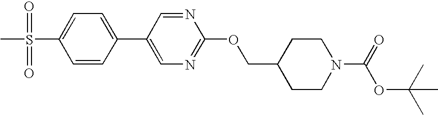

5-Bromo-2-chloropyrimidine (500 mg, 2.58 mmol), potassium tert-butoxide (348.1 mg, 3.10 mmol) and tert-butyl 4-(hydroxymethyl)piperidine-1-carboxylate (556.5 mg, 2.58 mmol) in dry THF (10 mL) were heated at 60° C. for 72 h. The reaction mixture was filtered and the solvent was removed under reduced pressure to give crude Intermediate A1. 4-Methylsulfonylphenylboronic acid (567.7 mg, 2.84 mmol), K₂CO₃ (1.43 g, 10.32 mmol) and [(C₆H₅)₃P]₄Pd (0.06 g, 0.05 mmol) in dioxane (5 mL) and water (1 mL) were added to the crude Intermediate A1 obtained above. The reaction mixture was heated at 90° C. for 16 h. The mixture was filtered and concentrated under reduced pressure. The residue was purified by preparative HPLC (System F, gradient 35-90% MeCN) to give the title compound. Yield 20 mg (2%). Analytical HPLC: purity 99% (System A, R_{T}=2.231 min); ¹H NMR (400 MHz, CD₃OD) δ ppm 1.13-1.31 (m, 3H) 1.38 (s, 9H) 1.75 (s, 1H) 1.91-2.11 (m, 2H) 2.73 (s, 2H) 3.08 (s, 3H) 4.05 (m, 2H) 4.25 (m, 2H) 7.79-7.92 (m, 2H) 7.92-8.08 (m, 2H) 8.84 (s, 2H); LRESIMS for C₂₂H₂₉N₃O₅S m/z 448 (+H)⁺.

### Example A3

### Benzyl 4-[({5-[4-(methylsulfonyl)phenyl]pyrimidin-2-yl}oxy)methyl]piperidine-1-carboxylate

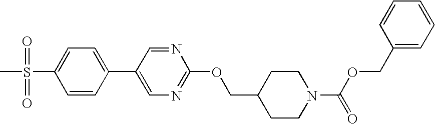

To a stirred solution of tert-butyl 4-[({5-[4-(methylsulfonyl)phenyl]pyrimidin-2-yl}oxy)methyl]piperidine-1-carboxylate (18.0 mg, 0.04 mmol; obtained in Example A2) in DCM (2 mL) was added TFA (0.5 mL). The reaction was stirred at ambient temperature for 1 h. The solution was distributed equally to two new vials. To one of the vials benzyl chloroformate (2.9 μL, 0.02 mmol) and triethylamine (8.4 μL, 0.06 mmol) were added and the mixture was shaken at ambient temperature for 16 h. The solvent was removed under reduced pressure and the residue was purified by preparative HPLC (System D, gradient 35-90% MeCN) to give the title compound. Yield 1.2 mg (12%). Analytical HPLC: purity 100% (System A, R_{T}=2.36 min); ¹H NMR (400 MHz, CD₃OD) δ ppm 1.26 (m, 2H) 1.78 (d, J=10.8 Hz, 2H) 2.81 (s, 2H) 3.08 (s, 3H) 4.12 (m, 2H) 4.25 (m, 2H) 4.51 (s, 1H) 5.04 (s, 2H) 7.17-7.33 (m, 5H) 7.79-7.89 (m, 2H) 7.93-8.04 (m, 2H) 8.84 (s, 2H); LRESIMS for C₂₅H₂₇N₃O₅S m/z 482 (M+H)⁺; HRESIMS calc. monoiso mass (Da): 481.1671, found monoiso mass (Da): 481.1669.

### Example A4

### 2-[(1-Benzoylpiperidin-4-yl)methoxy]-5-[4-(methylsulfonyl)phenyl]pyrimidine

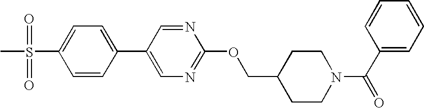

To a stirred solution of tert-butyl 4-[({5-[4-(methylsulfonyl)phenyl]pyrimidin-2-yl}oxy)-methyl]piperidine-1-carboxylate (18.0 mg, 0.04 mmol; obtained in Example A2) in DCM (2 mL) was added TFA (0.5 mL). The reaction was stirred at ambient temperature for 1 h. The solution was distributed equally to two new vials. To one of the vials benzoyl chloride (2.3 μL, 0.02 mmol) and triethylamine (8.4 μL, 0.06 mmol) were added and the mixture was shaken at ambient temperature for 16 h. The solvent was removed under reduced pressure and the residue was purified by preparative HPLC (System D, gradient 35-90% MeCN) to give the title compound. Yield 2.4 mg (27%). Analytical HPLC: purity 99% (System A, R_{T}=2.02 min); ¹H NMR (400 MHz, CD₃OD) δ ppm 1.33 (s, 2H) 1.74 (s, 1H) 1.95 (s, 2H) 2.05-2.21 (m, 1H) 2.76-2.93 (m, 1H) 3.08 (s, 3H) 3.69 (s, 1H) 4.29 (d, J=6.0 Hz, 2H) 4.60 (s, 1H) 7.28-7.46 (m, 5H) 7.80-7.89 (m, 2H) 7.93-8.03 (m, 2H) 8.85 (s, 2H); LRESIMS for C₂₄H₂₅N₃O₄S m/z 452 (M+H)⁺; HRESIMS calc. monoiso mass (Da): 451.1566, found monoiso mass (Da): 451.1562.

### Example A5

### tert-Butyl 4-{[(5-{4-[(methoxycarbonyl)amino]phenyl}pyrimidin-2-yl)oxy]methyl}-piperidine-1-carboxylate

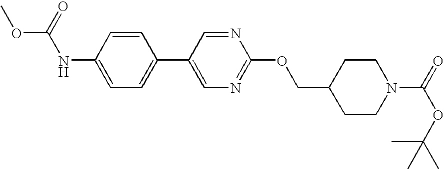

This compound was prepared from (4-methoxycarbonylaminophenyl)boronic acid using the conditions described in general method A1. Purified by preparative HPLC (System D, gradient 30-70% MeCN). Yield 19 mg (54%). Analytical HPLC: purity 96% (System A, R_{T}=2.38 min); ¹H NMR (400 MHz, CDCl₃) δ ppm 1.20-1.37 (m, 2H), 1.45 (s, 9H), 1.81-1.90 (m, 2H), 2.74 (m, 2H), 3.21 (s, 3H), 4.06-4.21 (m, 3H), 4.25 (d, J=6.5 Hz, 2H), 6.69 (s, 1H), 7.40-7.55 (m, 4H), 8.66 (s, 2H); LRESIMS for C₂₃H₃₀N₄O₅ m/z 443 (M+H)⁺; HRESIMS calc. monoiso mass (Da): 442.2216, found monoiso mass (Da): 442.2220.

### Example A6

### tert-Butyl 4-{1-[(5-{4-[(dimethylamino)carbonyl]phenyl}pyrimidin-2-yl)oxy]methyl}-piperidine-1-carboxylate

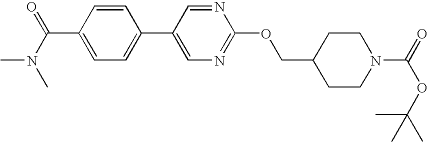

This compound was prepared from [4-(N,N-dimethylaminocarbonyl)phenyl]boronic acid using the conditions described in general method A2. Purified by preparative HPLC (System D, gradient 30-70% MeCN). Yield 19 mg (54%). Analytical HPLC: purity 100% (System A, R_{T}=2.25 min); ¹H NMR (400 MHz, CDCl₃) δ ppm 1.22-1.37 (m, 2H), 1.45 (s, 9H), 1.79-1.91 (m, 2H), 2.03 (m, 1H), 2.74 (m, 2H), 3.02 (s, 3H), 3.13 (s, 3H), 4.14 (br s, 2H), 4.25 (d, J=6.5 Hz, 2H), 7.50-7.57 (m, 4H), 8.71 (s, 2H); LRESIMS for C₂₄H₃₂N₄O₄ m/z 441 (M+H)⁺; HRESIMS calc. monoiso mass (Da): 440.2424, found monoiso mass (Da): 440.2431.

### Example A7

### tert-Butyl 4-[({5-[4-(acetylamino)phenyl]pyrimidin-2-yl}oxy)methyl]piperidine-1-carboxylate

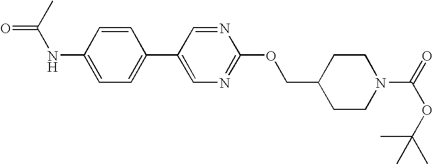

This compound was prepared from 4-acetamidophenyl boronic acid using the conditions described in general method A1. Purified by preparative HPLC (System D, gradient 30-70% MeCN). Yield 12 mg (35%). Analytical HPLC: purity 100% (System A, R_{T}=2.22 min); ¹H NMR (400 MHz, CDCl₃) δ ppm 1.21-1.37 (m, 2H), 1.45 (s, 9H), 1.80-1.90 (m, 2H), 2.03 (m, 1H), 2.20 (s, 3H), 2.74 (m, 2H), 4.04-4.21 (br s, 2H), 4.25 (d, J=6.5 Hz, 2H), 7.46 (m, 2H), 7.62 (m, 2H), 8.67 (s, 2H); LRESIMS for C₂₃H₃₀N₄O₄ m/z 427 (M+H)⁺; HRESIMS calc. monoiso mass (Da): 426.2267, found monoiso mass (Da): 426.2268.

### Example A8

### tert-Butyl 4-{[(5-{4-[(diethylamino)carbonyl]phenyl}pyrimidin-2-yl)oxy]methyl}-piperidine-1-carboxylate

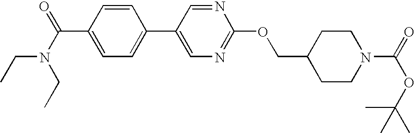

This compound was prepared from [4-(N,N-diethylaminocarbonyl)phenyl]boronic acid using the conditions described in general method A2. Purified by preparative HPLC (System D, gradient 30-70% MeCN). Yield 22 mg (59%). Analytical HPLC: purity 100% (System A, R_{T}=2.45 min); ¹H NMR (400 MHz, CDCl₃) δ ppm 1.07-1.38 (m, 9H), 1.44-1.47 (m, 10H), 1.80-1.91 (m, 2H), 2.03 (m, 1H), 2.75 (m, 2H), 3.30 (br s, 1H), 3.56 (br s, 1H), 4.06-4.21 (m, 2H), 4.27 (m, 2H), 7.45-7.57 (m, 4H), 8.70 (s, 2H); LRESIMS for C₂₆H₃₆N₄O₄ m/z 469 (M+H)⁺; HRESIMS calc. monoiso mass (Da): 468.2737, found monoiso mass (Da): 468.2740.

### Example A9

### tert-Butyl 4-[({5-[4-(aminocarbonyl)phenyl]pyrimidin-2-yl}oxy)methyl]piperidine-1-carboxylate

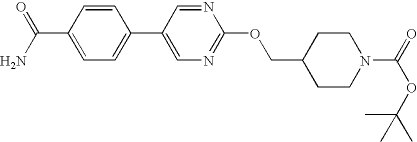

This compound was prepared from (4-aminocarbonylphenyl)boronic acid using the conditions described in general method A1. Purified by preparative HPLC (System D, gradient 25-65% MeCN). Yield 15 mg (45%). Analytical HPLC: purity 100% (System A, R_{T}=2.09 min); ¹H NMR (400 MHz, CDCl₃) δ ppm 1.14-1.30 (m, 2H), 1.37 (s, 9H), 1.71-1.83 (m, 2H), 1.97 (m, 1H), 2.68 (m, 2H), 4.04-4.05 (m, 2H), 4.20 (m, 2H), 7.53 (m, 2H), 7.88 (m, 2H), 8.66 (s, 2H); LRESIMS for C₂₂H₂₈N₄O₄ m/z 468 (M+H)⁺; HRESIMS calc. monoiso mass (Da): 468.2737, found monoiso mass (Da): 468.2740.

### Example A10

### tert-Butyl 4-{[(5-{4-[(methylsulfonyl)amino]phenyl}pyrimidin-2-yl)oxy]methyl}-piperidine-1-carboxylate

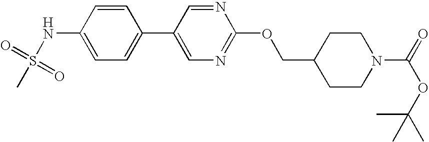

This compound was prepared from (4-methylsulfonylaminophenyl)boronic acid using the conditions described in general method A1 but without the preparative HPLC. Yield 19 mg (51%). Analytical HPLC: purity 96% (System A, R_{T}=2.25 min); ¹H NMR (400 MHz, CDCl₃) δ ppm 1.21-1.38 (m, 2H), 1.45 (s, 9H), 1.79-1.92 (m, 2H), 2.03 (m, 1H), 2.74 (m, 2H), 3.06 (s, 3H), 3.69 (s, 1H), 4.07-4.17 (br s, 2H), 4.26 (d, J=6.5 Hz, 2H), 6.60 (s, 1H), 7.33 (m, 2H), 7.50 (m, 2H), 8.67 (s, 2H); LRESIMS for C₂₂H₃₀N₄O₅S m/z 463 (M+H)⁺; HRESIMS calc. monoiso mass (Da): 462.1937, found monoiso mass (Da): 462.1932.

### Example A11

### tert-Butyl 4-[({5-[4-(morpholin-4-ylcarbonyl)phenyl]pyrimidin-2-yl}oxy)methyl]-piperidine-1-carboxylate

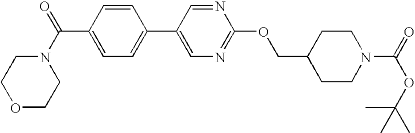

This compound was prepared from 4-(morpholinyl-4-carbonylphenyl)boronic acid using the conditions described in general method A2. To the crude product was added MeOH (1 mL). The precipitate was collected by filtration to give the pure product. Yield 9 mg (23%). Analytical HPLC: purity 100% (System A, R_{T}=2.22 min); ¹H NMR (400 MHz, CDCl₃) δ ppm 1.22-1.37 (m, 2H), 1.45 (s, 9H), 1.80-1.90 (m, 2H), 2.03 (m, 1H), 2.74 (m, 2H), 3.38-3.91 (m, 8H), 4.15 (bs, 2H), 4.27 (d, J=6.5 Hz, 2H), 7.49-7.59 (m, 4H), 8.70 (s, 2H); LRESIMS for C₂₆H₃₄N₄O₅ m/z 483 (M+H)⁺; HRESIMS calc. monoiso mass (Da): 482.2529, found monoiso mass (Da): 482.2574.

### Example A12

### tert-Butyl 4-[({5-[4-(aminosulfonyl)phenyl]pyrimidin-2-yl}oxy)methyl]piperidine-1-carboxylate

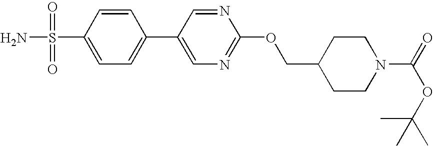

This compound was prepared from 4-boronobenzenesulfonamide using the conditions described in general method A1. Purified by preparative HPLC (System D, gradient 25-65% MeCN). Yield 2.8 mg (8%). Analytical HPLC: purity 100% (System A, R_{T}=2.14 min); ¹H NMR (400 MHz, CDCl₃-CD₃OD) Selected peaks: δ ppm 1.17-1.32 (m, 2H), 1.40 (s, 9H), 1.74-1.85 (m, 2H), 2.01 (m, 1H), 2.61-2.89 (m, obscured in part by solvent signal), 4.08 (m, 2H), 4.23 (d, J=6.5 Hz, 2H), 7.61 (m, 2H), 7.97 (m, 2H), 8.68 (s, 2H); LRESIMS for C₂₁H₂₈N₄O₅S m/z 393 (M+H-tBu)⁺; HRESIMS calc. monoiso mass (Da): 448.1780, found monoiso mass (Da): 448.1779.

### Example A13

### tert-Butyl 4-{[(5-{4-[(2-hydroxyethyl)sulfonyl]phenyl}pyrimidin-2-yl)oxy]methyl}-piperidine-1-carboxylate

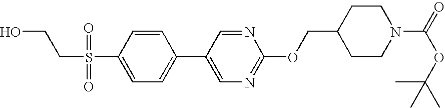

This compound was prepared from 2-[(4-bromophenyl)sulfonyl]ethanol using the conditions described in general method B. Yield 44 mg (46%). Analytical HPLC: purity 100% (System A, R_{T}=2.07 min); ¹H NMR (400 MHz, CD₃OD) δ ppm 1.22-1.36 (m, 2H), 1.84 (m, 2H), 2.07 (m, 1H), 2.81 (m, 2H), 3.45 (t, J=6 Hz, 2H), 3.89 (t, J=6 Hz, 2H), 4.13 (m, 2H), 4.33 (d, J=6.5 Hz, 2H), 7.92 (m, 2H), 8.04 (m, 2H), 8.92 (s, 2H); LRESIMS for C₂₃H₃₁N₃O₆S m/z 422 (M+H-t-Bu)⁺; HRESIMS found monoiso mass (Da): 477.1926 calc. monoiso mass (Da): 477.1934. The starting material, 2-[(4-bromophenyl)sulfonyl]-ethanol, was prepared using similar conditions as described in the literature procedure (Verhart, C. G. J et al., New base-labile amino-protective groups for peptide synthesis; Rec. Trav. Chim. Pays-Bas. 1988, 107(11), 621-6).

### Example A14

### tert-Butyl 4-[({5-[4-(morpholin-4-ylsulfonyl)phenyl]pyrimidin-2-yl}oxy)methyl]-piperidine-1-carboxylate

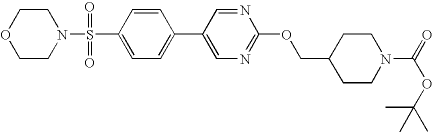

This compound was prepared from (4-morpholinosulfonylphenyl)boronic acid using the conditions described in general method A2. Purified by preparative HPLC (System D, gradient 30-70% MeCN). Yield 11 mg (26%). Analytical HPLC: purity 100% (System A, R_{T}=2.41 min); ¹H NMR (400 MHz, CDCl₃) δ ppm 1.22-1.38 (m, 2H), 1.45 (s, 9H), 1.80-1.91 (m, 2H), 2.05 (m, 1H), 2.75 (m, 2H), 3.04 (m, 4H), 3.76 (m, 4H), 4.17 (br s, 2H), 4.29 (d, J=6.5 Hz, 2H), 7.69 (m, 2H), 7.86 (m, 2H), 8.74 (s, 2H); LRESMS for C₂₅H₃₄N₄O₆S m/z 463 (M+H-tBu)⁺; HRESMS calc. monoiso mass (Da): 518.2199, found monoiso mass (Da): 518.2211.

### Example A15

### tert-Butyl 4-{[(5-{4-[(2,5-dioxoimidazolidin-1-yl)methyl]phenyl}pyrimidin-2-yl)oxy]-methyl}piperidine-1-carboxylate

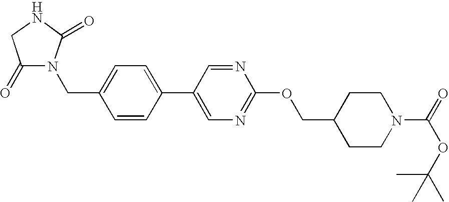

tert-Butyl 4-[({5-[4-(hydroxymethyl)phenyl]pyrimidin-2-yl}oxy)methyl]piperidine 1-carboxylate (Example A1; 80 mg, 0.20 mmol), triphenylphosphine (68 mg, 0.26 mmol) and hydantoin (26 mg, 0.26 mmol) were mixed with THF (2 mL) and N,N,N′,N′-tetramethylazodicarboxamide (46 mg, 0.26 mmol) was added. The mixture was stirred at r.t. overnight and the solvent was evaporated. Flash chromatography using 3% MeOH in CHCl₃ as eluent gave the title product. Yield 14 mg (29%). Analytical HPLC: purity 100% (System A, R_{T}=2.36 min); ¹H NMR (400 MHz, CDCl₃) δ ppm 1.20-1.36 (m, 2H), 1.45 (s, 9H), 1.80-1.90 (m, 2H), 2.03 (m, 1H), 2.74 (m, 2H), 3.99 (s, 2H), 4.15 (m, 2H), 4.25 (d, J=6.5 Hz, 2H), 4.71 (s, 2H), 5.50 (s, 1H), 7.43-7.55 (m, 4H), 8.67 (s, 2H); LRESIMS for C₂₅H₃₁N₅O₅ m/z 482 (M+H)⁺; HRESIMS calc. monoiso mass (Da): 481.2325, found monoiso mass (Da): 481.2325.

### Example A16

### tert-Butyl 4-{[(5-{4-[(dimethylamino)sulfonyl]phenyl}pyrimidin-2-yl)oxy]methyl}-piperidine-1-carboxylate

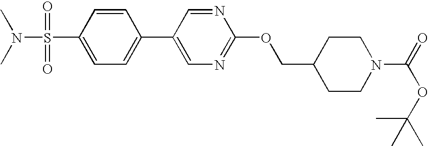

This compound was prepared from N,N-dimethyl-4-boronobenzenesulfonamide using the conditions described in general method A2. Purified by preparative HPLC (System D, gradient 25-65% MeCN). Yield 12 mg (31%). Analytical HPLC: purity 100% (System A, R_{T}=2.46 min); ¹H NMR (400 MHz, CDCl₃) δ ppm 1.22-1.37 (m, 2H), 1.45 (s, 9H), 1.80-1.90 (m, 2H), 2.04 (m, 1H), 2.68-2.81 (m, 8H), 4.17 (m, 2H), 4.28 (d, J=6.5 Hz, 2H), 7.68 (m, 2H), 7.88 (m, 2H), 8.74 (s, 2H); LRESIMS for C₂₃H₃₂N₄O₅S m/z 477 (M+H)⁺; HRESIMS calc. monoiso mass (Da): 476.2093, found monoiso mass (Da): 476.2099.

### Example A17

### tert-Butyl 4-[({5-[4-({[2-(dimethylamino)ethyl]amino}carbonyl)phenyl]pyrimidin-2-yl}oxy)methyl]piperidine-1-carboxylate

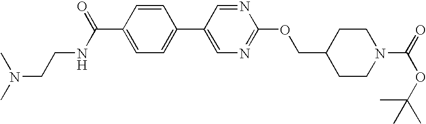

This compound was prepared from 4-[{2-(N,N-dimethylamino)ethyl}aminocarbonyl]-phenylboronic acid using the conditions described in general method A2. Purified by preparative HPLC (System D, gradient 25-65% MeCN). Yield 24 mg (62%). Analytical HPLC: purity 100% (System A, R_{T}=1.92 min); ¹H NMR (400 MHz, CDCl₃-CD₃OD) Selected peaks: 6 ppm 1.13-1.30 (m, 2H), 1.37 (s, 9H), 1.71-1.82 (m, 2H), 2.51 (s, 6H), 2.68 (m, 2H), 2.88 (m, 2H), 4.05 (m, 2H), 4.20 (d, J=6.5 Hz, 2H), 7.52 (m, 2H), 7.90 (m, 2H), 8.66 (s, 2H); LRESIMS for C₂₆H₃₇N₅O₄ m/z 484 (M+H)⁺; HRESIMS calc. monoiso mass (Da): 483.2846, found monoiso mass (Da): 483.2844.

### Example A18

### tert-Butyl 4-[({5-[4-(aminocarbonyl)-3-fluorophenyl]pyrimidin-2-yl}oxy)methyl]-piperidine-1-carboxylate

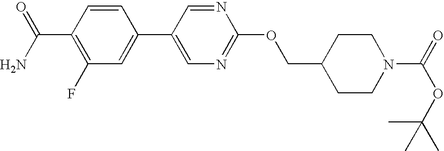

This compound was prepared from 4-carbamoyl-3-fluoro-phenylboronic acid using the conditions described in general method A1. Analytical HPLC: purity 99% (System A, R_{T}=2.11 min); ¹H NMR (400 MHz, CD₃OD) δ ppm 1.24-1.34 (m, 2H) 1.45 (s, 9H) 1.82 (m, 2H) 2.01-2.11 (m, 1H) 2.80 (br s, 2H) 4.13 (m, 2H) 4.32 (d, J=6.5 Hz, 2H) 7.57-7.61 (m, 2H) 7.94 (m, 1H) 8.90 (s, 2H); LRESIMS for C₂₂H₂₇FN₄O₄ m/z 431 (M+H)⁺. HRESIMS, calc. monoiso mass (Da): 430.2016, found monoiso mass (Da): 430.2005.

### Example A19

### Isopropyl 4-[({5-[4-(methylsulfonyl)phenyl]pyrimidin-2-yl}amino)methyl]piperidine-1-carboxylate

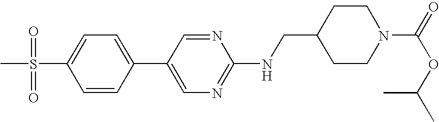

5-[4-(Methylsulfonyl)phenyl]-N-(piperidin-4-ylmethyl)pyrimidin-2-amine (Intermediate A3; 260 mg, 0.75 mmol) was mixed with DCM (7.7 mL) and Et₃N (0.275 mL, 2.00 mmol). 0.725 mL (˜0.066 mmol) of this mixture was added to isopropyl chloroformate (0.1 mL of a 1 M solution in toluene, 0.1 mmol). The mixture was stirred at r.t. for 5 h and then a 2 M solution of NH₃ in MeOH (0.1 mL) was added. The volatiles were evaporated in vacuo and MeOH was added to the residue. The precipitate was collected by filtration to give the crude product. Yield 17 mg (60%). Analytical HPLC: purity 98% (System A, R_{T}=1.96 min); ¹H NMR (400 MHz, CDCl₃) δ ppm 1.13-1.28 (m, 8H), 1.71-1.88 (m, 3H), 2.73 (m, 2H), 3.20 (s, 3H), 3.40 (t, J=6.3 Hz, 2H), 4.16 (br s, 2H), 4.90 (m, 1H), 5.39 (t, J=6.2 Hz, 1H), 7.66 (m, 2H), 8.00 (m, 2H), 8.54 (s, 2H); LRESIMS for C₂₁H₂₈N₄O₄S m/z 433 (M+H)⁺. HRESIMS calc. monoiso mass (Da): 432.1831, found monoiso mass (Da): 432.1836.

### Example A20

### Ethyl 4-[({5-[4-(methylsulfonyl)phenyl]pyrimidin-2-yl}amino)methyl]piperidine-1-carboxylate, trifluoroacetate

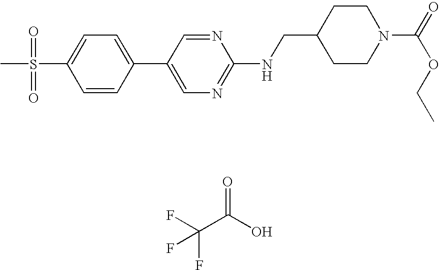

5-[4-(Methylsulfonyl)phenyl]-N-(piperidin-4-ylmethyl)pyrimidin-2-amine (Intermediate A3; 260 mg, 0.75 mmol) was mixed with DCM (7.7 mL) and Et₃N (0.275 mL, 2.00 mmol). 0.725 mL (−0.066 mmol) of this mixture was added to ethyl chloroformate (11 mg, 0.1 mmol) and the mixture was stirred at r.t. overnight. Then a 2 M solution of NH₃ in MeOH (0.1 mL) was added. The volatiles were evaporated in vacuo and the residue was purified by preparative HPLC (System D) to give the title compound. Yield 18 mg (51%). Analytical HPLC: purity 100% (System A, R_{T}=1.82 min); ¹H NMR (400 MHz, CD₃OD) δ ppm 1.19 (m, 2H), 1.24 (t, J=7.1 Hz, 3H), 3.11-3.16 (m, 3H), 2.80 (m, 2H), 3.14 (s, 3H), 3.36 (d, J=6.8 Hz, 2H), 4.05-4.17 (m, 4H), 7.84 (m, 2H), 8.01 (m, 2H), 8.68 (s, 2H); LRESIMS for C₂₀H₂₆N₄O₄S m/z 419 (M+H)⁺. HRESIMS calc. monoiso mass (Da): 418.1675, found monoiso mass (Da): 418.1676.

### Example A21

### N-{[1-(3,3-Dimethylbutanoyl)piperidin-4-yl]methyl}-5-[4-(methylsulfonyl)phenyl]-pyrimidin-2-amine, trifluoroacetate

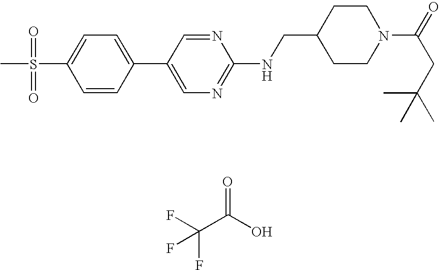

5-[4-(Methylsulfonyl)phenyl]-N-(piperidin-4-ylmethyl)pyrimidin-2-amine (Intermediate A3; 260 mg, 0.75 mmol) was mixed with DCM (7.7 mL) and Et₃N (0.275 mL, 2.00 mmol). 0.725 mL (˜0.066 mmol) of this mixture was added to tert-butylacetyl chloride (13 mg, 0.1 mmol). The mixture was stirred at r.t. for 5 h and a 2 M solution of NH₃ in MeOH (0.1 mL) was added. The volatiles were evaporated in vacuo and the residue was purified by preparative HPLC (System D) to give the title compound. Yield 22 mg (60%). Analytical HPLC: purity 100% (System A, R_{T}=1.95 min); ¹H NMR (400 MHz CD₃OD) δ ppm 1.03 (s, 9H), 1.1-1.6 (m, 2H), 1.85 (m, 2H), 1.96 (m, 1H), 2.27 (m, 1H), 2.38 (m 1H), 2.61 (m, 1H), 3.12 (m, 1H), 3.14 (s, 3H), 3.37 (d, J=7.0 Hz, 2H), 4.07 (m, 1H), 4.59 (m, 1H), 7.84 (m, 2H), 8.01 (m, 2H), 8.67 (s, 2H); LRESIMS for C₂₃H₃₂N₄O₃S m/z 445 (M+H)⁺. HRESIMS calc. monoiso mass (Da): 444.2195, found monoiso mass (Da): 444.2197.

### Example A22

### tert-Butyl 4-[({5-[4-(methylsulfonyl)phenyl]pyrimidin-2-yl}amino)methyl]piperidine-1-carboxylate

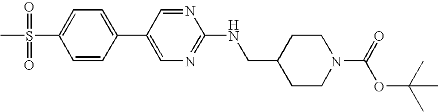

A suspension of 5-bromo-2-chloropyrimidine (0.50 g, 2.58 mmol), K₂CO₃ (0.43 g, 3.10 mmol) and tert-butyl 4-(aminomethyl)piperidine-1-carboxylate (0.55 g, 2.58 mmol) in DMF (20 mL) was heated at 110° C. for 16 h. The reaction mixture was filtered and the solvent was removed under reduced pressure to give crude tert-butyl 4-{[(5-bromo-pyrimidine-2-yl)amino]methyl}piperidine-1-carboxylate.

4-Methylsulfonylphenylboronic acid (0.57 g, 2.84 mmol), K₂CO₃ (1.43 g, 10.32 mmol) and [(C₆H₅)₃P]₄Pd (0.06 g, 0.05 mmol) in dioxane (5 mL) and water (1 mL) were added to the crude tert-butyl 4-{[(5-bromopyrimidine-2-yl)amino]methyl}piperidine-1-carboxylate. The suspension was heated at 90° C. for 16 h, filtered and concentrated under reduced pressure. The residue was purified by preparative HPLC (System F, gradient 35-90% MeCN) to give the title compound. Yield 157.9 mg (14%). Analytical HPLC: purity 100% (System A, R_{T}=2.12 min); ¹H NMR (400 MHz, CD₃OD) δ ppm 1.05-1.21 (m, 2H) 1.42 (s, 9H) 1.73 (s, 2H) 1.79-1.92 (m, 1H) 2.72 (s, 1H) 3.07-3.14 (s, 3H) 3.30-3.35 (m, 3H) 4.06 (m, 2H) 7.77-7.85 (m, 2H) 7.93-8.03 (m, 2H) 8.62 (s, 2H); LRESIMS for C₂₂H₃₀N₄O₄S m/z 447 (M+H)⁺; HRESIMS calc. monoiso mass (Da): 446.1988, found monoiso mass (Da): 446.1978.

### Example A23

### Benzyl 4-[({5-[4-(methylsulfonyl)phenyl]pyrimidin-2-yl}amino)methyl]piperidine-1-carboxylate

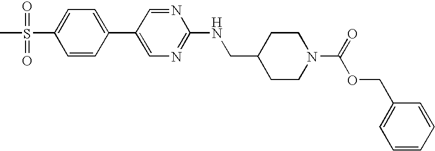

TFA (0.5 mL) was added to a stirred solution tert-butyl 4-[({5-[4-(methylsulfonyl)phenyl]-pyrimidin-2-yl}amino)methyl]piperidine-1-carboxylate (152 mg, 0.34 mmol; obtained in Example A22) in DCM (2 mL). The reaction mixture was stirred at ambient temperature for 16 h. The solvent was removed under reduced pressure. To the crude mixture (10.0 mg, 0.029 mmol) were added DCM (1 mL), benzyl chloroformate (4.1 μL, 0.029 mmol) and triethylamine (12.1 μL, 0.087 mmol). The reaction was shaken at ambient temperature for 16 h. The solvent was removed under reduced pressure and the residue was purified by preparative HPLC (System E) using acetonitrile-water gradients containing 0.1% ammonium acetate to give the title compound. Yield 1.5 mg (11%). Analytical HPLC: purity 97% (System A and B, R_{TA}=2.49 min, R_{TB}=2.34 min); ¹H NMR (400 MHz, CD₃OD) δ ppm 1.16 (s, 2H) 1.65-1.96 (m, 3H) 3.07-3.17 (m, 3H) 3.29-3.38 (m, 4H) 4.12 (s, 2H) 5.08 (s, 2H) 7.15-7.47 (m, 5H) 7.74-7.93 (m, 2H) 7.90-8.06 (m, 2H) 8.62 (s, 2H); LRESIMS for C₂₅H₂₈N₄O₄S m/z 481 (M+H)⁺; HRESIMS, calc. monoiso mass (Da): 480.1831, found monoiso mass (Da): 480.1839.

### Example A24

### tert-Butyl 4-({[5-(4-{[(2R)-2-(hydroxymethyl)pyrrolidin-1-yl]carbonyl}phenyl) -pyrimidin-2-yl]oxy}methyl)piperidine-1-carboxylate

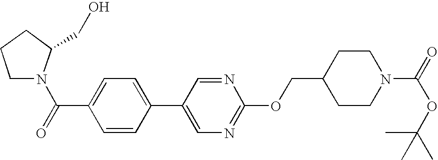

4-(2-{[1-(tert-Butoxycarbonyl)piperidin-4-yl]methoxy}pyrimidin-5-yl)benzoic acid (Inter-mediate A2; 58 mg, 0.14 mmol) was dissolved in DMF (0.8 mL) and Et₃N (0.025 mL, 0.18 mmol). (R)-(−)-2-(hydroxymethyl)pyrrolidine (18 mg, 0.18 mmol) was added and then TBTU (56 mg, 0.174 mmol). The reaction mixture was stirred at r.t. overnight, evaporated in vacuo and the residue was purified by preparative HPLC (System E, gradient 30-60% MeCN) to give the title compound. Yield 35 mg (50%). Analytical HPLC: purity 100% (System A, R_{T}=2.18 min); ¹H NMR (400 MHz, CD₃OD) 5 ppm 1.22-1.36 (m, 2H), 1.46 (s, 9H), 1.70-1.89 (m, 3H), 1.90-2.17 (m, 4H), 2.81 (m, 2H), 3.40-3.64 (m, 2H), 3.73-3.89 (m, 2H), 4.07-4.17 (m, 2H), 4.26-4.36 (m, 3H), 7.67 (m, 2H), 7.74 (m, 2H), 8.86 (s, 2H); LRESIMS for C₂₇H₃₆N₄O₅ m/z 497 (M+H)⁺; HRESIMS calc. monoiso mass (Da): 496.2686, found monoiso mass (Da): 496.2678.

### Example A25

### tert-Butyl 4-{[(5-{4-[(2,5-dioxopyrrolidin-1-yl)methyl]phenyl}pyrimidin-2-yl)oxy]-methyl}piperidine-1-carboxylate

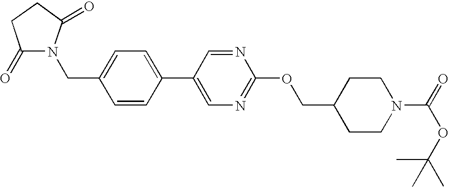

To a mixture of tert-butyl 4-[({5-[4-(hydroxymethyl)phenyl]pyrimidin-2-yl}oxy)methyl]-piperidine-1-carboxylate (Example A1; 80 mg, 0.2 mmol), triphenylphosphine (68 mg, 0.26 mmol) and succinimide (26 mg, 0.26 mmol) in THF (2 mL) was added N,N,N′,N′-tetramethylazodicarboxamide (46 mg, 0.26 mmol). The mixture was stirred at r.t. overnight and the solvent was evaporated under reduced pressure. The residue was purified by preparative HPLC (System D, gradient 31-61% MeCN) to give the title compound. Yield 11 mg (26%). Analytical HPLC: purity 98% (System A, R_{T}=2.21 min); ¹H NMR (400 MHz, CDCl₃) δ ppm 1.21-1.37 (m, 2H), 1.45 (s, 9H), 1.59-1.74 (br s, 1H), 1.78-1.90 (m, 2H), 2.03 (m, 1H), 2.68-2.80 (m, 6H), 4.08-4.20 (m, 2H), 4.25 (d, J=6.5 Hz, 2H), 4.70 (s, 2H), 7.41-7.53 (m, 4H), 8.62-8.69 (m, 2H); LRESIMS for C₂₆H₃₂N₄O₅ m/z 481 (M+H)⁺; HRESIMS calc. monoiso mass (Da): 480.2373, found monoiso mass (Da): 480.2368.

### Example A26

### tert-Butyl 4-{[(5-{4-[(4-methylpiperazin-1-yl)carbonyl]phenyl}pyrimidin-2-yl)oxy]-methyl}piperidine-1-carboxylate

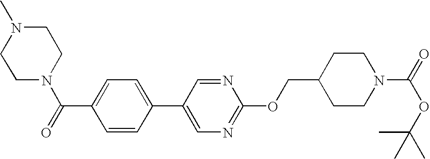

4-(2-{[1-(tert-Butoxycarbonyl)piperidin-4-yl]methoxy}pyrimidin-5-yl)benzoic acid (Inter-mediate A2; 58 mg, 0.14 mmol) was dissolved in DMF (0.8 mL) and Et₃N (0.025 mL, 0.18 mmol). N-Methylpiperazine (19 mg, 0.19 mmol) and TBTU (56 mg, 0.174 mmol) were added. The reaction mixture was stirred at r.t. overnight, concentrated in vacuo and the residue was purified by preparative HPLC (System E, gradient 30-60% MeCN) to give the title compound. Yield 38 mg (55%). Analytical HPLC: purity 100% (System A, R_{T}=1.84 min); ¹H NMR (400 MHz, CD₃OD) δ ppm 1.21-1.36 (m, 2H), 1.45 (s, 9H), 1.78-1.89 (m, 2H), 2.07 (m, 1H), 2.33 (s, 3H), 2.36-2.91 (m, 6H), 3.42-3.60 (bs, 2H), 3.71-3.87 (br s, 2H), 4.08-4.17 (m, 2H), 4.32 (d, J=6.6 Hz, 2H), 7.55 (m, 2H), 7.75 (m, 2H), 8.86 (s, 2H); LRESIMS for C₂₇H₃₇N₅O₄ m/z 496 (M+H)⁺; HRESIMS calc. monoiso mass (Da): 495.2846, found monoiso mass (Da): 495.2843.

### Example A27

### tert-Butyl 4-({[5-(3,5-difluoro-4-hydroxyphenyl)pyrimidin-2-yl]oxy}methyl) -piperidine-1-carboxylate

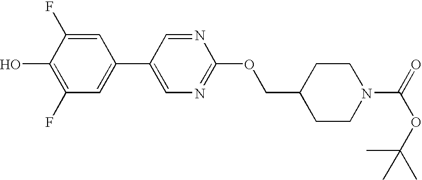

This compound was prepared from 4-bromo-2,6-difluorophenol using the conditions described in general method B. The combined fractions were evaporated and the residue was dissolved in 5% MeOH/CHCl₃ and passed through a silica gel plug using 5% MeOH in CHCl₃ as eluent. Yield 10 mg (12%). Analytical HPLC: purity 100% (System A, R_{T}=2.40 min); ¹H NMR (400 MHz, CD₃OD) δ ppm 1.21-1.34 (m, 2H), 1.45 (s, 9H), 1.83 (m, 2H), 2.04 (m, 1H), 2.80 (m, 2H), 4.12 (m, 2H), 4.28 (d, J=6.5 Hz, 2H), 7.20-7.31 (m, 2H), 8.75 (s, 2H); LRESIMS for C₂₁H₂₅F₂N₃O₄ m/z 422 (M+H)⁺; HRESIMS, calc. monoiso mass (Da): 421.1813, found monoiso mass (Da): 421.1810.

### Example A28

### tert-Butyl 4-({[5-(4-{[3-(dimethylamino)pyrrolidin-1-yl]carbonyl}phenyl)pyrimidin-2-yl]oxy}methyl)piperidine-1-carboxylate

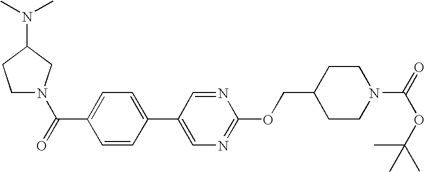

To a solution of 4-(2-{[1-(tert-butoxycarbonyl)piperidin-4-yl]methoxy}pyrimidin-5-yl)-benzoic acid (Intermediate A2; 58 mg, 0.14 mmol) in DMF (0.8 mL) and Et₃N (0.025 mL, 0.18 mmol) were added 3-(dimethylamino)pyrrolidine (20 mg, 0.175 mmol) and TBTU (56 mg, 0.174 mmol). The reaction mixture was stirred at r.t. overnight, evaporated in vacuo and the residue was purified by preparative HPLC (System E, gradient 28-58% MeCN) to give the title compound. Yield 41 mg (40%). Analytical HPLC: purity 100% (System A, R_{T}=1.89 min); ¹H NMR (400 MHz, CD₃OD) Selected peaks: 8 ppm 1.22-1.36 (m, 2H), 1.46 (s, 9H), 1.80-1.89 (m, 2H), 1.91-2.14 (m, 3H), 2.25-2.43 (m, 4H), 2.68-3.15 (m, 8H), 4.08-4.17 (m, 2H), 4.32 (d, J=6.5 Hz, 2H), 7.45-7.57 (br s, 2H), 7.75 (m, 2H), 8.86 (s, 2H); LRESIMS for C₂₈H₃₉N₅O₄ m/z 510 (M+H)⁺; HRESIMS, calc. monoiso mass (Da): 509.3002, found monoiso mass (Da): 509.3004.

### Example A29

### tert-Butyl 4-{[(5-{3-fluoro-4-[(propylamino)carbonyl]phenyl}pyrimidin-2-yl)oxy]-methyl}piperidine-1-carboxylate

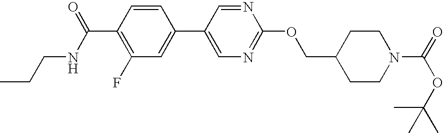

This compound was prepared from 3-fluoro-4-(N-propylcarbamoyl)phenylboronic acid using the conditions described in general method A1. Analytical HPLC: purity 100% (System A, R_{T}=2.50 min); ¹H NMR (400 MHz, CD₃OD) δ ppm 0.99 (t, J=7.4 Hz, 3H) 1.23-1.34 (m, 2H) 1.45 (s, 9H) 1.60-1.69 (m, 2H) 1.82 (m, 2H) 2.00-2.11 (m, 1H) 2.80 (br s, 2H) 3.36 (m, 2H) 4.13 (m, 2H) 4.32 (d, J=6.5 Hz, 2H) 7.55-7.60 (m, 2H) 7.81 (m, 1H) 8.89 (s, 2H); LRESIMS for C₂₅H₃₃FN₄O₄ m/z 473 (M+H)⁺; HRESIMS calc. monoiso mass (Da): 472.2486, found monoiso mass (Da): 472.2478.

### Example A30

### tert-Butyl 4-{[(5-{4-[(3-hydroxypyrrolidin-1-yl)carbonyl]phenyl}pyrimidin-2-yl)oxy]-methyl}piperidine-1-carboxylate

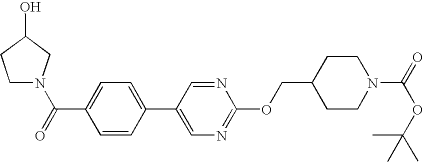

4-(2-{[1-(tert-butoxycarbonyl)piperidin-4-yl]methoxy}pyrimidin-5-yl)benzoic acid (Intermediate A2; 58 mg, 0.14 mmol) was dissolved in DMF (0.8 mL) and Et₃N (0.025 mL, 0.18 mmol). 3-pyrrolidinol (15 mg, 0.172 mmol) was added and then TBTU (56 mg, 0.174 mmol). The reaction mixture was stirred at r.t. overnight, evaporated in vacuo and the residue was purified by preparative HPLC (System E, gradient 25-55% MeCN) to give the title compound. Yield 34 mg (50%). Yield 34 mg (50%). Analytical HPLC: purity 99% (System A, R_{T}=2.02 min); ¹H NMR (400 MHz, CD₃OD) δ ppm 1.22-1.36 (m, 2H), 1.46 (s, 9H), 1.79-1.89 (m, 2H), 1.97-2.18 (m, 3H), 2.81 (m, 2H), 3.34-3.83 (m, 4H), 4.08-4.17 (m, 2H), 4.32 (d, J=6.5 Hz, 2H), 4.38 (s, 0.5H), 4.50 (s, 0.5H), 7.62-7.70 (m, 2H), 7.75 (m, 2H), 8.86 (s, 2H); LRESIMS for C₂₆H₃₄N₄O₅ m/z 483 (M+H)⁺; HRESIMS, calc. monoiso mass (Da): 482.2529, found monoiso mass (Da): 482.2524.

### Intermediate B1

### 5-Methyl-2-[4-(methylsulfonyl)phenyl]pyrimidine

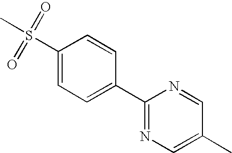

A suspension of 2-chloro-5-methyl-pyrimidine (4.0 g, 0.0311 mol), (4-methylsulfonyl)-phenylboronic acid (7.47 g, 0.0373 mol), Pd(PPh₃)₄ (1.8 g, 0.0016 mol) and potassium carbonate (17.2 g, 0.125 mol) in 1,4-dioxane (50 mL) and water (40 mL) was heated at reflux overnight. The mixture was allowed to reach r.t. and then concentrated under reduced pressure. The residue was partitioned between water (30 mL) and chloroform (50 mL). The layers were separated and the water phase was extracted with chloroform (2×50 mL). The combined organic phases were combined and dried over potassium carbonate, filtered and concentrated. The crude product was purified by flash chromatography on silica using EtOAc/petroleum ether (1:1) as eluent. Yield 4.44 g (58%). Analytical HPLC: purity 95% (System A, R_{T}=1.64 min); LRESIMS for C₁₂H₁₂N₂O₂S m/z 249 (M+H)⁺.

### Intermediate B2

### 5-(Chloromethyl)-2-[4-(methylsulfonyl)phenyl]pyrimidine

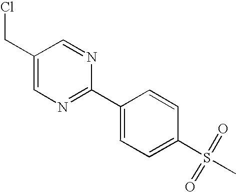

To a flask containing 5-methyl-2-[4-(methylsulfonyl)phenyl]pyrimidine (4.44 g, 0.0179 mol) in chloroform (100 mL) was added N-chlorosuccinimide (4.78 g, 0.0358 mol) and benzoyl peroxide (0.434 g, 0.0018 mol). The mixture was stirred for 96 hours, with two further additions of N-chlorosuccinimide (1 equiv each) and benzoyl peroxide (0.1 equiv each), and then evaporated. The crude product was used without further purification in the subsequent step (Example B1).

### Example B1

### tert-Butyl 4-[({2-[4-(methylsulfonyl)phenyl]pyrimidin-5-yl}methyl)amino]piperidine-1-carboxylate

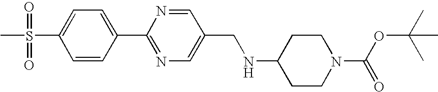

A suspension of 5-(chloromethyl)-2-[4-(methylsulfonyl)phenyl]pyrimidine (Intermediate B2; 130 mg, 0.46 mmol), tert-butyl 4-aminopiperidine-1-carboxylate (92 mg, 0.46 mmol), N,N-diisopropylethylamine (0.2 mL, 1.15 mmol) in MeCN (2 mL) was heated at 60° C. overnight. The mixture was concentrated and the residue was purified by preparative HPLC (System E, gradient 18-48% MeCN). Yield 32 mg (16%); Analytical HPLC: purity 100% (System A, R_{T}=1.59 min); ¹H NMR (400 MHz, CD₃OD) δ ppm 1.25-1.39 (m, 2H) 1.48 (s, 9H) 1.93-2.02 (m, 2H) 2.69-2.78 (m, 1H) 2.79-2.93 (m, 2H) 3.20 (s, 3H) 3.92 (s, 2H) 4.02-4.12 (m, 2H) 8.06-8.13 (m, 2H) 8.65-8.72 (m, 2H) 8.90-8.94 (m, 2H); LRESIMS for C₂₂H₃₀N₄O₄S m/z 447 (M+H)⁺; HRESIMS, calc. monoiso mass (Da): 446.1988, found monoiso mass (Da): 446.1985.

### Example B2

### tert-Butyl 4-[methyl({2-[4-(methylsulfonyl)phenyl]pyrimidin-5-yl}methyl)amino]-piperidine-1-carboxylate

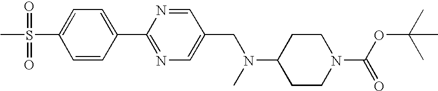

To a mixture of tert-butyl 4-[({2-[4-(methylsulfonyl)phenyl]pyrimidin-5-yl}methyl)-amino]piperidine-1-carboxylate (15 mg, 0.034 mmol; obtained in Example B1) and NaBH(OAc)₃ (14 mg, 0.068 mmol) in methanol (1.5 mL) was added a 37% solution of formalin (3 μL, 0.050 mmol). The mixture was stirred overnight, evaporated and purified by preparative HPLC (System E, gradient 20-60% MeCN). Yield 5 mg (32%); Analytical HPLC: purity 100% (System A, R_{T}=1.64 min); ¹H NMR (400 MHz, CDCl₃) δ ppm 1.40 (s, 9H) 1.41-1.52 (m, 2H) 1.70-1.79 (m, 2H) 2.17 (s, 3H) 2.49-2.70 (m, 3H) 3.03 (s, 3H) 3.59 (s, 2H) 4.05-4.20 (m, 2H) 7.96-8.02 (m, 2H) 8.56-8.62 (m, 2H) 8.72 (s, 2H); LRESIMS for C₂₃H₃₂N₄O₄S m/z 461 (M+H)⁺;)⁺; HRESIMS, calc. monoiso mass (Da): 460.2144, found monoiso mass (Da): 460.2143.

Biological Tests

Human GPR119 Activity Assay

Agonists to the human GPR119 receptor were characterized by measuring human GPR119 receptor-mediated stimulation of cyclic AMP (cAMP) in HEK 293 cells expressing the human GPR119 receptor.

Briefly, cAMP content was determined using a cAMP kit based on HTRF technology (Homogeneous Time-Resolved Fluorescence, Cisbio Cat. no. 62AM2PEC). HEK293 cells stably expressing the human GPR119 receptor (HEK293-hGPR119 cells) were cultured in DMEM (Gibco # 31966-021) supplemented with 10% Bovine Calf Serum (Hyclone # SH30072.03), and, 500 μg/mL Hygromycin B (Roche Diagnostics 843555). At 80% confluency, cells were detached using Trypsine and aliquoted at a density of 5×10⁶ cells/mL in freezing medium (DMEM (Gibco # 31966-021), 20% BCS (Hyclone # SH30072.03), 10% DMSO (Sigma #D2650) and stored at −135° C. On the experimental day, HEK293-hGPR119 cells were thawn and diluted to 0.4×10⁶ cells/mL in assay buffer (1×HBSS (Gibco Cat. no. 14025-049), 20 mM Hepes (Gibco Cat. no. 15630-056), 0.1% BSA, pH 7.4) and incubated with test substances for 20 min at room temperature. After addition of HTRF reagents diluted in lysis buffer, the 96- or 384-well plates were incubated 1 hour, followed by measuring the fluorescence ratio at 665 nm/620 nm. Test substances was diluted in compound buffer (1×HBSS (Gibco Cat. no. 14025-049), 20 mM Hepes (Gibco Cat. no. 15630-056), 0.1% BSA, 2 mM IBMX (Sigma-Aldrich Cat. No. 17018, pH 7.4). The potency of the agonist was quantified by determine the concentration that cause 50% activation of hGPR119 evoked increase in cAMP, EC₅₀.

Compounds of the invention showed a concentration-dependant increase in intracellular cAMP level and generally had an EC₅₀ value of <5 μM. Obtained EC₅₀ values for representative compounds of the present invention are shown in Table A.

TABLE A
Agonist potency at the human GPR119.

| Compound | EC₅₀ (μM) |
|---|---|
| EXAMPLE A2 | 0.014 |
| EXAMPLE A22 | 0.055 |
| EXAMPLE B1 | 0.453 |

Effects of GPR119 Modulators on Glucose-Stimulated Insulin Release
In Vitro Experiments

The effect of GPR119 modulators on glucose-stimulated insulin release is determined in isolated pancreatic islets from Wistar rats and diabetic rat models, e.g. GK rat. Briefly, islets are isolated from the rats by digestion with collagenase according to standard protocol. The islets are cultured for 24 h in RPMI-1640 medium supplemented with 11.1 mM glucose and 10% (vol/vol) fetal calf serum. On the experimental day, batches of three islets are preincubated in KRB (Krebs-Ringer bicarbonate) buffer and 3.3 mM glucose for 30 min, 37° C. Thereafter the batches with islets are incubated in 16.7 mM glucose and KRB buffer supplemented with vehicle or test compounds for 60 min at 37° C. Aliquots of the medium will be frozen for measurement of insulin using a radioimmunoassay with rabbit ant-porcine insulin antibodies.

In Vivo Experiments

The effects of GPR119 modulators on glucose stimulated insulin release is determined in diabetic mice models (eg. Lep^{ob/ob} or diet-induced obese (DIO) mice) undergoing an oral glucose tolerance test. Briefly, overnight fasted mice is given either vehicle or test compound at desired doses via oral gavage. Based on the pharmacokinetic of the test compounds, a glucose boluse dose is delivered via oral gavage 30 min-2 hrs following the test compound. Plasma glucose and insulin levels are determined at desired time points over a 2 hour period using blood collection from tail nick. Plasma glucose is determined using a Glucometer and plasma insulin is determined using an insulin ELISA following blood collection in heparinated tubes and centrifugation.

Effects of GPR119 Modulators on GLP-1 Secretion and Body Weight

In Vivo Experiments

The effect of GPR119 modulators on body weight is determined in diabetic and obese mice models, eg. Lep^{ob/ob} or diet-induced obese (DIO) mice. The food intake and body weight gain is measured during subchronic treatment with vehicle or test compound via oral gavage. At the end of the experiment, vena cava blood is collected and e.g. HbA1c, GLP-1, insulin, ALAT, ASAT are measured.

1. A compound of Formula (Ia) 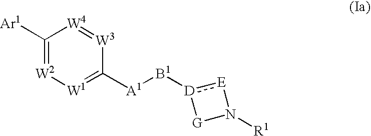
including pharmaceutically acceptable salts, hydrates, geometrical isomers, racemates, tautomers, optical isomers, and N-oxides thereof; wherein:
W¹ and W³ are N and W² and W⁴ are CR¹², or W¹ and W³ are CR¹² and W² and W⁴ are N;
A¹ is CH₂, O, NR¹⁰, S, S(O) or S(O)₂;
B¹ is CH₂, O, NR¹⁰, S, S(O), S(O)₂, C(O) or CONR¹⁰, provided that when B′ is O,
NR¹⁰, S, S(O), S(O)₂, C(O) or CONR¹⁰, then A¹ is CH₂;
D is N, C or CR¹¹, provided that D must be CR¹¹ and said R¹¹ must be hydrogen or methyl when B¹ is selected from O, NR¹⁰, S, S(O), S(O)₂, and CONR¹⁰; [symbol]
is a single bond when D is N or CR¹¹ or a double bond when D is C;
E and G are independently C₁₋₃-alkylene, each optionally independently substituted with a substituent selected from the group consisting of C₁₋₃-alkyl, C₁₋₄-alkoxy, carboxy, fluoro-C₁₋₃-alkyl, hydroxy, hydroxymethyl, and fluoro, provided that the ring formed by D, E, N and G has not more than 7 ring atoms, and further provided that the said ring has 6 or 7 ring atoms when D is N, and yet further provided that the total number of substituents on E and G independently is not more than 2;
R¹ is C(O)OR², C(O)R², S(O)₂R², C(O)NR²R³, —CH₂—C(O)NR²R³, or a 5- or 6-membered heteroaryl group linked via a ring carbon atom, wherein the said heteroaryl group is optionally substituted with C₁₋₄-alkyl;
Ar¹ is phenyl which is optionally substituted in one or more positions with a substituent independently selected from:
(a) CF₃SO₃,
(b) halogen selected from chlorine, bromine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —CH₂—NR⁶C(O)R⁴,
(h) —NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) CH₃OC(O)NH—,
(p) C-heterocyclyl, optionally substituted with C₁₋₄-alkyl,
(q) —CN,
(r) —OR⁸,
(s) —SCF₃,
(t) —NO₂,
(u) phosphonooxy,
(v) C-heterocyclylsulfonyl, optionally substituted with C₁₋₄-alkyl,
(w) —NR⁵R⁵,
(x) —C(OH)CH₃CF₃,
(y) [C(OH)CH₃CF₃]—C₁₋₆-alkyl,
(z) cyano-C₁₋₆-alkyl,
(aa) guanidino,
(bb) amidino,
(cc) C₁₋₆-alkyl,
(dd) C₁₋₄-alkoxy-C₁₋₄-alkyl,
(ee) fluoro-C₁₋₄-alkyl,
(ff) C₂₋₆-alkenyl,
(gg) fluoro-C₂₋₄-alkenyl,
(hh) hydroxy-C₁₋₆-alkyl,
(ii) C₁₋₄-alkylsulfonyl-C₁₋₄-alkyl,
(jj) hydroxy-C₂₋₄-alkoxy-C₁₋₄-alkyl,
(kk) C₂₋₃-acyl-C₁₋₃-alkyl,
(ll) C₂₋₆-alkynyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with C₁₋₄-alkyl,
(qq) C₃₋₆-cycloalkyl,
(rr) C₃₋₆-cycloalkyl-C₁₋₄-alkyl,
(ss) R⁵R⁵N—C₁₋₂-alkyl,
(tt) —C(O)OR⁷,
(uu) aryl,
(vv) aryl-C₁₋₄-alkyl,
(ww) aryl-C₂₋₄-alkenyl,
(xx) aryl-C₂₋₄-alkynyl,
(yy) heteroaryl,
(zz) heteroaryl-C₁₋₄-alkyl,
(aaa) heteroaryl-C₂₋₄-alkenyl, and
(bbb) heteroaryl-C₂₋₄-alkynyl,
wherein any aryl or heteroaryl residue, alone or as part of another group, as substituent on Ar¹ is optionally substituted in one or more positions with a substituent independently selected from the group Z¹ consisting of:
(a) halogen selected from chlorine and fluorine,
(b) C₁₋₄-alkyl,
(c) hydroxy,
(d) C₁₋₄-alkoxy,
(e) —OCF₃,
(f) —SCF₃,
(g) —CN,
(h) —C(OH)CH₃CF₃,
(i) hydroxy-C₁₋₄-alkyl,
(i) —CF₃,
(k) —S(O)₂CH₃,
(l) —S(O)₂NH₂,
(m) —S(O)₂NHCH₃,
(n) —S(O)₂N(CH₃)₂,
(o) —N(CH₃)S(O)₂CH₃,
(p) —N(CH₃)C(O)CH₃,
(q) —C(O)NH₂,
(r) —C(O)NHCH₃,
(s) —C(O)N(CH₃)₂,
(t) —C(O)CH₃,
(u) —NH₂,
(v) —NHCH₃,
(w) —N(CH₃)₂,
(x) —NO₂, and
(y) methoxycarbonyl;
R² is selected from:
(a) C₁₋₆-alkyl,
(b) C₁₋₆-alkoxy-C₂₋₆-alkyl,
(c) hydroxy-C₂₋₆-alkyl,
(d) fluoro-C₂₋₆-alkyl,
(e) C₃₋₆-alkynyl,
(f) C₃₋₆-alkenyl,
(g) C₃₋₇-cycloalkyl,
(h) C₅₋₈-cycloalkenyl,
(i) NR⁹R⁹, provided that R¹ is not selected from C(O)OR², C(O)NR²R³ and —CH₂—C(O)NR²R³,
(j) C-heterocyclyl, optionally substituted with C₁₋₄-alkyl,
(k) C₇₋₈-bicyclyl, optionally substituted with hydroxy,
(l) C₇₋₈-bicyclylmethyl,
(m) azabicyclyl, optionally substituted with hydroxy,
(n) C₃₋₇-cycloalkyl-C₁₋₄-alkyl, wherein cycloalkyl is optionally substituted with methyl,
(o) C₁₋₆-alkylsulfonyl-C₂₋₆-alkyl,
(p) C₂₋₃-acyl-C₁₋₄-alkyl,
(q) arylcarbonyl-C₁₋₄-alkyl,
(r) heteroarylcarbonyl-C₁₋₄-alkyl,
(s) [C(OH)CH₃CF₃]—C₁₋₆-alkyl,
(t) N-heterocyclylcarbonyl-C₂₋₄-alkyl, wherein heterocyclyl is optionally substituted with methyl,
(u) C-heterocyclylcarbonyl-C₂₋₄-alkyl, wherein heterocyclyl is optionally substituted with methyl,
(v) aminocarbonyl-C₂₋₆-alkyl,
(w) C₁₋₃-alkylaminocarbonyl-C₂₋₆-alkyl,
(x) di(C₁₋₃-alkyl)aminocarbonyl-C₂₋₆-alkyl,
(y) hydroxy-C₂₋₄-alkoxy-C₂₋₄-alkyl,
(z) hydroxy-C₄₋₆-cycloalkyl,
(aa) oxo-C₄₋₆-cycloalkyl,
(bb) fluoro-C₄₋₆-cycloalkyl,
(cc) C₁₋₃-alkoxy-C₄₋₆-cycloalkyl,
(dd) methyl-C₃₋₆-cycloalkyl,
(ee) oxo-N-heterocyclyl-C₂₋₄-alkyl,
(ff) fluoro-N-heterocyclyl-C₂₋₄-alkyl,
(gg) amino-N-heterocyclyl-C₂₋₄-alkyl,
(hh) hydroxy-N-heterocyclyl-C₂₋₄-alkyl,
(ii) N-heterocyclyl-C₂₋₄-alkyl, wherein heterocyclyl is optionally substituted with methyl,
(jj) C-heterocyclyl-C₁₋₄-alkyl, wherein heterocyclyl is optionally substituted with methyl,
(kk) aryl,
(ll) aryl-C₁₋₄-alkyl,
(mm) aryl-C₃₋₆-alkenyl,
(nn) aryl-C₃₋₆-alkynyl,
(o) heteroaryl,
(pp) heteroaryl-C₁₋₄-alkyl,
(qq) heteroaryl-C₃₋₆-alkenyl, and
(rr) heteroaryl-C₃₋₆-alkynyl,
wherein any aryl or heteroaryl residue, alone or as part of another group, is optionally independently substituted in one or more position with a substituent selected from the group Z¹;
R³ is selected from:
(a) hydrogen,
(b) C₁₋₆-alkyl,
(c) fluoro-C₂₋₆-alkyl,
(d) hydroxy-C₂₋₆-alkyl,
(e) C₁₋₆-alkoxy-C₂₋₆-alkyl,
(f) amino-C₂₋₆-alkyl,
(g) C₁₋₃-alkylamino-C₂₋₆-alkyl,
(h) di(C₁₋₃-alkyl)amino-C₂₋₆-alkyl,
(i) cyano-C₁₋₆-alkyl, and
(j) C₁₋₆-alkylsulfonyl-C₂₋₆-alkyl;
R⁴ is independently selected from:
(a) C₁₋₆-alkyl,
(b) fluoro-C₁₋₆-alkyl,
(c) hydroxy-C₂₋₆-alkyl,
(d) C₁₋₄-alkoxy-C₂₋₄-alkyl,
(e) C₂₋₄-acyl-C₁₋₄-alkyl,
(f) carboxy-C₁₋₃-alkyl,
(g) C₃₋₆-cycloalkyl,
(h) oxo-C₄₋₆-cycloalkyl,
(i) hydroxy-C₄₋₆-cycloalkyl,
(j) fluoro-C₄₋₆-cycloalkyl,
(k) methyl-C₃₋₆-cycloalkyl,
(l) N-heterocyclylcarbonyl-C₂₋₄-alkyl, wherein heterocyclyl is optionally substituted with methyl,
(m) oxo-N-heterocyclyl-C₂₋₄-alkyl,
(n) fluoro-N-heterocyclyl-C₂₋₄-alkyl,
(o) hydroxy-N-heterocyclyl-C₂₋₄-alkyl,
(p) amino-N-heterocyclyl-C₂₋₄-alkyl,
(q) aminocarbonyl-C₂₋₄-alkyl,
(r) C₁₋₃-alkylaminocarbonyl-C₂₋₄-alkyl,
(s) di(C₁₋₃-alkyl)aminocarbonyl-C₂₋₄-alkyl,
(t) C₂₋₃-acylamino-C₂₋₄-alkyl,
(u) hydroxy-C₂₋₄-alkoxy-C₂₋₄-alkyl,
(v) C-heterocyclylcarbonyl-C₂₋₄-alkyl, wherein heterocyclyl is optionally substituted with methyl,
(w) C₃₋₆-cycloalkyl-C₁₋₂-alkyl,
(x) aryl,
(y) aryl-C₁₋₂-alkyl,
(z) heteroaryl, and
(aa) heteroaryl-C₁₋₂-alkyl,
wherein any aryl or heteroaryl residue, alone or as part of another group, is optionally substituted in one or more positions with a substituent independently selected from the group Z² consisting of:
(a) halogen selected from chlorine and fluorine,
(b) C₁₋₄-alkoxy,
(c) hydroxymethyl,
(d) —CN,
(e) —CF₃,
(f) C₁₋₄-alkyl,
(g) —OCF₃, and
(h) —C(O)CH₃;
R⁵ is each independently selected from:
(a) hydrogen,
(b) C₁₋₆-alkyl,
(c) C₃₋₄-cycloalkyl,
(d) fluoro-C₂₋₄-alkyl,
(e) amino-C₂₋₆-alkyl,
(f) cyano-C₁₋₆-alkyl,
(g) hydroxy-C₂₋₆-alkyl,
(h) dihydroxy-C₂₋₆-alkyl,
(i) C₁₋₄-alkoxy-C₂₋₄-alkyl,
(j) C₁₋₄-alkylamino-C₂₋₄-alkyl,
(k) di(C₁₋₄-alkyl)amino-C₂₋₄-alkyl,
(l) aminocarbonyl-C₁₋₄-alkyl,
(m) C₂₋₃-acylamino-C₂₋₄-alkyl,
(n) C₁₋₄-alkylthio-C₂₋₄-alkyl,
(o) C₂₋₄-acyl-C₁₋₄-alkyl, and
(p) C₁₋₄-alkylsulfonyl-C₁₋₄-alkyl, or
two R⁵ groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁵ groups form a piperazine ring, the nitrogen of the piperazine ring that allows the substitution is optionally substituted with C₁₋₄-alkyl;
R⁶ is independently selected from:
(a) hydrogen,
(b) C₁₋₄-alkyl, and
(c) hydroxy-C₂₋₄-alkyl;
R⁷ is independently selected from:
(a) hydrogen, and
(b) C₁₋₄-alkyl;
R⁸ is independently selected from:
(a) hydrogen,
(b) C₁₋₆-alkyl,
(c) fluoro-C₁₋₆-alkyl,
(d) hydroxy-C₂₋₆-alkyl,
(e) amino-C₂₋₆-alkyl,
(f) C₁₋₃-alkylamino-C₂₋₄-alkyl,
(g) di(C₁₋₃-dialkyl)amino-C₂₋₄-alkyl,
(h) C₁₋₄-alkylsulfonyl-C₂₋₄-alkyl,
(i) N-heterocyclyl-C₂₋₄-alkyl, wherein heterocyclyl is optionally substituted with methyl,
(j) C-heterocyclyl, optionally substituted with methyl,
(k) C₂₋₃-acylamino-C₂₋₄-alkyl,
(l) [C(OH)CH₃CF₃]—C₁₋₆-alkyl,
(m) C₃₋₆-cycloalkyl,
(n) methyl-C₃₋₆-cycloalkyl,
(o) C₃₋₆-cycloalkyl-C₁₋₂-alkyl,
(p) aryl, and
(q) heteroaryl,
wherein any aryl or heteroaryl residue is optionally independently substituted in one or two positions with a substituent selected from the group Z²;
R⁹ is each independently selected from:
(a) C₁₋₄-alkoxy-C₂₋₄-alkyl,
(b) amino-C₂₋₄-alkyl,
(c) C₁₋₄-alkylamino-C₂₋₄-alkyl,
(d) di(C₁₋₄-alkyl)amino-C₂₋₄-alkyl,
(e) C₂₋₃-acylamino-C₂₋₄-alkyl,
(f) C₁₋₄-alkylthio-C₂₋₄-alkyl, and
(g) C₂₋₄-acyl-C₁₋₄-alkyl,
or two R⁹ groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁹ groups form a piperazine ring, the nitrogen of the piperazine ring that allows the substitution is optionally substituted with C₁₋₄-alkyl;
R¹⁰ is independently selected from:
(a) hydrogen,
(b) C₁₋₆-alkyl,
(c) cyclopropyl,
(d) cyclobutyl,
(e) cyclopropylmethyl,
(f) fluoro-C₂₋₆-alkyl,
(g) hydroxy-C₂₋₆-alkyl,
(h) C₁₋₂-alkoxy-C₂₋₆-alkyl,
(i) amino-C₂₋₆-alkyl,
(j) di(C₁₋₃-alkyl)amino-C₂₋₆-alkyl,
(k) C₁₋₃-alkylamino-C₂₋₆-alkyl,
(l) cyano-C₁₋₄-alkyl,
(m) C₂₋₆-acyl,
(n) C₂₋₆-acyl-C₁₋₆-alkyl,
(o) C₁₋₆-alkylsulfonyl-C₁₋₆-alkyl, and
(p) tetrahydrofuran-2-ylmethyl;
R¹¹ is selected from:
(a) hydrogen,
(b) hydroxy,
(c) fluorine,
(d) C₁₋₄-alkoxy, and
(e) methyl;
R¹² is each independently selected from:
(a) hydrogen,
(b) halogen selected from chlorine and fluorine,
(c) —S(O)₂CH₃,
(d) —S(O)₂CF₃,
(e) —OS(O)₂CF₃,
(f) —S(O)NH₂,
(g) —S(O)₂NHCH₃,
(h) —S(O)₂N(CH₃)₂,
(i) —NHS(O)₂CH₃,
(j) —N(CH₃)S(O)₂CH₃,
(k) —NHC(O)CH₃,
(l) —N(CH₃)C(O)CH₃,
(m) —C(O)NH₂,
(n) —C(O)NHCH₃,
(o) —C(O)N(CH₃)₂,
(p) —CN,
(q) —CF₃,
(r) guanidino,
(s) amidino,
(t) —OH,
(u) C₁₋₄-alkoxy,
(v) —OCF₃,
(w) C₃₋₅-cycloalkyloxy,
(x) —SCF₃,
(y) —NO₂,
(z) —NR⁵R⁵, wherein each R⁵ is independently selected from the group consisting of hydrogen and C₁₋₄-alkyl; or two R⁵ groups together with the nitrogen to which they are attached form a pyrrolidine or an azetidine ring,
(aa) —C(OH)CH₃CF₃,
(bb) C₁₋₃-alkyl,
(cc) C₁₋₃-alkoxy-C₁₋₂-alkyl,
(dd) C₂₋₃-acyl,
(ee) C₂₋₃-alkenyl,
(ff) hydroxy-C₁₋₄-alkyl,
(gg) fluoro-C₂₋₃-alkyl,
(hh) C₂₋₃-alkynyl, and
(ii) C₃₋₅-cycloalkyl.

2. A compound according to claim 1 having Formula (Ib) 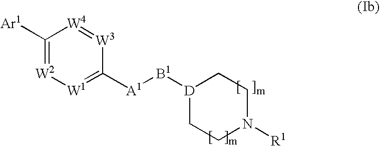
wherein
W¹ and W³ are N and W² and W⁴ are CR¹², or W¹ and W³ are CR¹² and W² and W⁴ are N;
A¹ is CH₂, O, NR¹⁰, S, S(O) or S(O)₂;
B¹ is CH₂, O, NR¹⁰, S, S(O), S(O)₂, C(O) or CONR¹⁰, provided that when B¹ is O, NR¹⁰, S, S(O), S(O)₂, C(O) or CONR¹⁰, then A¹ is CH₂;
m is each independently 0 or 1;
D is N or CR¹¹, provided that D must be CR¹¹ and said R¹¹ must be hydrogen or methyl when B¹ is selected from O, NR¹⁰, S, S(O), S(O)₂, and CONR¹⁰, and further provided that each m is 1 when D is N;
Ar¹, Z¹, Z², R¹ to R⁹ and R¹² are as defined in claim 1;
R¹⁰ is independently selected from:
(a) hydrogen,
(b) C₁₋₄-alkyl,
(c) cyclopropyl,
(d) cyclobutyl,
(e) cyclopropylmethyl,
(f) fluoro-C₂₋₄-alkyl,
(g) C₁₋₂-alkoxy-C₂₋₃-alkyl,
(h) hydroxy-C₂₋₄-alkyl,
(i) C₂₋₃-acyl,
(j) amino-C₂₋₄-alkyl,
(k) methylamino-C₂₋₄-alkyl,
(l) dimethylamino-C₂₋₄-alkyl,
(m) cyano-C₁₋₄-alkyl, and
(n) tetrahydrofuran-2-ylmethyl;
R¹¹ is selected from:
(a) hydrogen,
(b) hydroxy,
(c) fluorine, and
(d) methyl.

3. A compound according to claim 1 having Formula (Ic) 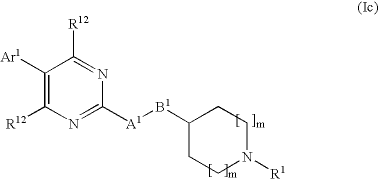
wherein A¹ is CH₂, O or NR¹⁰;
B¹ is CH₂, O or NR¹⁰, provided that when B¹ is O or NR¹⁰, then A¹ is CH₂;
m is each independently 0 or 1;
Z¹, Z², R⁷ to R¹, R⁹ and R¹² are as defined in claim 1, provided that at least one of R¹² is hydrogen;
R¹⁰ is as defined in claim 2;
Ar¹ is phenyl, which is optionally substituted in one, two or three positions with a substituent independently selected from the group Z³ consisting of:
(a) CF₃SO₃,
(b) halogen selected from bromine, chlorine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —NR⁶C(O)R⁴,
(h) —CH₂—NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O,
(o) —NHC(O)OCH₃,
(p) C-heterocyclyl, optionally substituted with methyl,
(q) —CN,
(r) —OR⁸,
(s) —SCF₃,
(t) —NO₂,
(u) phosphonooxy,
(v) C-heterocyclylsulfonyl, optionally substituted with methyl,
(w) —NR⁵R⁵,
(x) —C(OH)CH₃CF₃,
(y) cyano-C₁₋₆-alkyl,
(z) guanidino,
(aa) amidino,
(bb) C₁₋₆-alkyl,
(cc) C₁₋₄-alkoxy-C₁₋₄-alkyl,
(dd) fluoro-C₁₋₄-alkyl,
(ee) C₂₋₆-alkenyl,
(ff) fluoro-C₂₋₄-alkenyl,
(gg) hydroxy-C₁₋₆-alkyl,
(hh) C₁₋₄-alkylsulfonyl-C₁₋₄-alkyl,
(ii) hydroxy-C₂₋₄-alkoxy-C₁₋₄-alkyl,
(j) C₂₋₃-acyl-C₁₋₃-alkyl,
(kk) C₂₋₆-alkynyl,
(ll) C₃₋₆-cycloalkyl,
(mm) hydroxy-C₃₋₆-cycloalkyl,
(nn) fluoro-C₃₋₆-cycloalkyl,
(o) methyl-C₃₋₆-cycloalkyl,
(pp) C-heterocyclylcarbonyl, optionally substituted with methyl,
(qq) C₃₋₆-cycloalkyl-C₁₋₄-alkyl,
(rr) R⁵R⁵N—C₁₋₂-alkyl,
(ss) —C(O)OR⁷,
(tt) aryl, and
(uu) heteroaryl,
wherein any aryl or heteroaryl residue as substituent on Ar¹ is optionally substituted in one or more positions with a substituent independently selected from the group Z¹ as defined in claim 1;
R⁸ is independently selected from:
(g) hydrogen,
(h) C₁₋₄-alkyl,
(i) CF₃,
(j) C₃₋₅-cycloalkyl,
(k) methyl-C₃₋₅-cycloalkyl, and
(l) C-heterocyclyl, optionally substituted with methyl.

4. A compound according to claim 3, wherein
A¹ is CH₂ and B¹ is O or NR¹⁰, or
A¹ is A or NR¹⁰ and B¹ is CH₂; and
m is each 1.

5. A compound according to claim 4, wherein
Ar¹ is phenyl, which is optionally substituted in one, two or three positions with a substituent independently selected from the group Z⁴ consisting of:
(a) halogen selected from chlorine and fluorine,
(b) C₁₋₄-alkylsulfonyl,
(c) C₁₋₄-alkylsulfinyl,
(d) hydroxy-C₂₋₄-alkylsulfonyl,
(e) C₃₋₅-cycloalkylsulfonyl,
(f) methyl-C₃₋₅-cycloalkylsulfonyl,
(g) trifluoromethylsulfonyl,
(h) —S(O)₂NR^{5A}R^{5A},
(i) C₁₋₄-alkylsulfonamido,
(j) C₂₋₄-acylamino,
(k) C₂₋₄-acylaminomethyl,
(l) carboxy-C₁₋₃-alkylcarbonylamino,
(m) —C(O)NR^{5A}R^{5A},
(n) —CH₂—C(O)NR^{5A}R^{5A}
(o) —NHC(O)OCH₃,
(p) C₂₋₄-acyl,
(q) C₃₋₅-cycloalkylcarbonyl,
(r) C₁₋₄-alkoxy,
(s) C₃₋₅-cycloalkyloxy,
(t) C-heterocyclyl,
(u) —CN,
(v) —OH,
(w) —OCF₃,
(x) —CF₃,
(y) —NO₂,
(aa) —C(OH)CH₃CF₃,
(bb) cyano-C₁₋₂-alkyl,
(cc) C₁₋₄-alkyl,
(dd) C₃₋₅-cycloalkyl,
(ee) C₁₋₂-alkoxy-C₁₋₂-alkyl,
(ff) vinyl,
(gg) ethynyl,
(hh) hydroxy-C₁₋₂-alkyl,
(ii) C-heterocyclyloxy, optionally substituted with methyl,
(kk) —C(O)OR^{7A};
R¹ is a group R^{1A} selected from C(O)OR^{2A}, C(O)R^{2A}, S(O)₂R², C(O)NR^{2A}R^{1A}, and —CH₂—C(O)NR^{2A}R^{3A};
R² is selected from:
(a) C₁₋₆-alkyl,
(b) C₁₋₆-alkoxy-C₂₋₆-alkyl,
(c) hydroxy-C₂₋₆-alkyl,
(d) hydroxy-C₂₋₄-alkoxy-C₂₋₄-alkyl,
(e) fluoro-C₂₋₆-alkyl,
(f) C₃₋₆-alkynyl,
(g) C₃₋₇-cycloalkyl,
(h) C₅₋₈-cycloalkenyl,
(i) NR^{9A}R^{9A} provided that R^{1A} is not selected from C(O)OR^{2A}, C(O)NR^{2A}R^{3A} and —CH₂—C(O)NR^{2A}R^{3A},
(j) C-heterocyclyl, optionally substituted with methyl,
(k) C₇₋₈-bicyclyl,
(l) 2-norbornylmethyl,
(m) azabicyclyl,
(n) C₃₋₆-cycloalkyl-C₁₋₄-alkyl, wherein cycloalkyl is optionally substituted with methyl
(o) C₂₋₃-acyl-C₁₋₄-alkyl,
(p) arylcarbonyl-C₁₋₄-alkyl,
(q) heteroarylcarbonyl-C₁₋₄-alkyl,
(r) [C(OH)CH₃CF₃]—C₁₋₆-alkyl,
(s) N-heterocyclylcarbonyl-C₂₋₄-alkyl, wherein heterocyclyl is optionally substituted with methyl,
(t) hydroxy-C₄₋₆-cycloalkyl,
(u) oxo-C₄₋₆-cycloalkyl,
(v) fluoro-C₄₋₆-cycloalkyl,
(w) methoxy-C₄₋₆-cycloalkyl,
(x) methyl-C₃₋₆-cycloalkyl,
(y) oxo-N-heterocyclyl-C₂₋₄-alkyl,
(z) hydroxy-N-heterocyclyl-C₂₋₄-alkyl,
(aa) fluoro-N-heterocyclyl-C₂₋₄-alkyl,
(bb) amino-N-heterocyclyl-C₂₋₄-alkyl,
(cc) N-heterocyclyl-C₂₋₄-alkyl, wherein heterocyclyl is optionally substituted with methyl,
(dd) C-heterocyclyl-C₁₋₄-alkyl, wherein heterocyclyl is optionally substituted with methyl,
(ee) aryl,
(ff) aryl-C₁₋₄-alkyl,
(gg) heteroaryl, and
(hh) heteroaryl-C₁₋₄-alkyl,
wherein any aryl or heteroaryl residue, alone or as apart of another group, is optionally independently substituted in one or more positions with a substituent selected from the group Z⁵ consisting of:
(a) halogen selected from chlorine and fluorine,
(b) methyl,
(c) ethyl,
(d) methoxy,
(e) ethoxy,
(f) isopropoxy,
(g) hydroxy,
(h) —OCF₃,
(i) —CF₃,
(j) —CN,
(k) —C(OH)CH₃CF₃,
(l) dimethylamino,
(m) hydroxymethyl,
(n) —S(O)₂CH₃,
(o) —(O)CH₃, and
(p) —C(O)NH₂;
R^{3A} is selected from:
(a) hydrogen,
(b) C₁₋₄-alkyl,
(c) hydroxy-C₂₋₄-alkyl, and
(d) methoxy-C₂₋₄-alkyl;
R^{5A} is each independently selected from:
(a) hydrogen,
(b) C₁₋₃-alkyl,
(c) C₁₋₂-alkoxy-C₂₋₄-alkyl,
(d) C₃₋₄-cycloalkyl,
(e) hydroxy-C₂₋₄-alkyl,
(f) cyano-C₁₋₃-alkyl,
(g) C₂₋₃-acylamino-C₂₋₃-alkyl,
(h) dihydroxy-C₂₋₄-alkyl,
(i) aminocarbonyl-C₁₋₂-alkyl, and
(j) di(C₁₋₂-alkyl)amino-C₂₋₃-alkyl, or
two R^{5A} groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R^{5A} groups form a piperazine ring, the nitrogen of the piperazine ring that allows the substitution is optionally substituted with methyl;
R^{7A} is independently from:
(a) hydrogen, and
(b) C₁₋₄-alkyl;
Two groups R^{9A} together with the nitrogen to which they are attached form a heterocyclic ring, wherein said heterocyclic ring may be optionally substituted with: i) one hydroxy or amino group, ii) one or two fluorine atoms, or iii) one or two oxo groups, provided that when the substituent is selected from fluorine, hydroxy and amino, said substituent is attached to the heterocyclic ring at a position other than alpha to a heteroatom; and when the two R^{9A} groups form a piperazine ring, the nitrogen of the piperazine ring that allows the substitution is optionally substituted with methyl;
R¹⁰ is independently selected from:
(a) hydrogen, and
(b) C₁₋₃-alkyl;
R¹² is each hydrogen.

6. A compound according to claim 5, wherein A¹ is O or NR¹⁰ and B¹ is CH₂.

7. A compound according to claim 5, wherein Ar¹ is selected from methylsulfonylphenyl, [(methoxycarbonyl)amino]phenyl, (dimethylamino)carbonyl-phenyl, (acetylamino)phenyl, [(diethylamino)carbonyl]phenyl, (aminocarbonyl)-phenyl, [(methylsulfonyl)amino]phenyl, (morpholin-4-ylcarbonyl)phenyl, (amino-sulfonyl)phenyl, [(2-hydroxyethyl)sulfonyl]phenyl, (morpholin-4-ylsulfonyl)phenyl, [(2,5-dioxoimidazolidin-1-yl)methyl]phenyl, [(dimethylamino)sulfonyl]phenyl, {[2-(dimethylamino)ethyl]aminocarbonyl}phenyl, {[(2-hydroxymethyl)pyrrolidin-1-yl]-carbonyl}phenyl, [(2,5-dioxopyrrolidin-1-yl)methyl]phenyl, [(4-methylpiperazin-1-yl)carbonyl]phenyl, (difluoro)hydroxyphenyl, fluoro-[(propylamino)carbonyl]-phenyl, (aminocarbonyl)fluorophenyl, {[3-(dimethylamino)pyrrolidin-1-yl]-carbonyl}phenyl[(3-hydroxypyrrolidin-1-yl)carbonyl]phenyl and (hydroxymethyl)-phenyl.

8. A compound according to claim 5, wherein R^{1A} is selected from C(O)OR^{2A} and C(O)R^{2A}.

9. A compound according to claim 5, wherein R^{1A} is C(O)OR^{2A} and wherein R^{2A} is selected from C₁₋₆-alkyl and benzyl.

10. A compound according to claim 5, wherein R^{1A} is C(O)R^{2A} and wherein R^{2A} is selected from C₁₋₆-alkyl and phenyl.

11. A compound according to claim 5, wherein R¹⁰ is independently selected from hydrogen and methyl.

12. A compound according to claim 1 having Formula (Id) 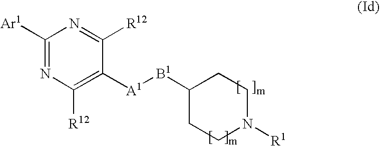
wherein A¹ is CH₂, O or NR¹⁰;
B¹ is CH₂, O or NR¹⁰, provided that when B¹ is O or NR¹⁰, then A¹ is CH₂;
m is each independently 0 or 1;
Z¹, Z², R¹ to R⁷, R⁹ and R¹² are as defined in claim 1, provided that at least one of R¹² is hydrogen;
R⁸ is as defined in claim 3;
R¹⁰ is as defined in claim 2;
Ar¹ is phenyl which is optionally substituted in one or two positions with a substituent independently selected from the group Z³ as defined in claim 3.

13. A compound according to claim 12, wherein
A¹ is CH₂ and B¹ is O or NR¹⁰, or
A¹ is or NR¹⁰ and B¹ is CH₂; and
m is each 1.

14. A compound according to claim 13, wherein
Ar¹ is phenyl, which is optionally substituted in one or two positions with a substituent independently selected from the group Z⁴ as defined in claim 5;
Z⁵ is as defined in claim 5;
R¹ is a group R^{1A}, wherein R^{1A} is as defined in claim 5;
R^{2A}, R^{3A}, R^{5A}, R^{7A} and R^{9A} are as defined in claim 5;
R¹⁰ is selected from hydrogen and C₁₋₃-alkyl;
R¹² is each hydrogen.

15. A compound according to claim 14 wherein A¹ is CH₂ and B¹ is NR¹⁰.

16. A compound according to claim 14, wherein Ar¹ is C₁₋₄-alkylsulfonylphenyl.

17. A compound according to claim 14, wherein R¹ is C(O)OR².

18. A compound according to claim 14, wherein R² is C₁₋₄-alkyl.

19. A compound according to claim 14, wherein R¹⁰ is independently selected from hydrogen, methyl and ethyl.

20. A compound according to claim 1, which is selected from:
tert-Butyl 4-[({5-[4-(hydroxymethyl)phenyl]pyrimidin-2-yl}oxy)methyl]-piperidine-1-carboxylate;
tert-Butyl 4-[({5-[4-(methylsulfonyl)phenyl]pyrimidin-2-yl}oxy)methyl]-piperidine-1-carboxylate;
Benzyl 4-[({5-[4-(methylsulfonyl)phenyl]pyrimidin-2-yl}oxy)methyl]-piperidine-1-carboxylate;
2-[(1-Benzoylpiperidin-4-yl)methoxy]-5-[4-(methylsulfonyl)phenyl]-pyrimidine;
tert-Butyl 4-{[(5-{4-[(methoxycarbonyl)amino]phenyl}pyrimidin-2-yl)oxy]-methyl}piperidine-1-carboxylate;
tert-Butyl 4-{[(5-{4-[(dimethylamino)carbonyl]phenyl}pyrimidin-2-yl)oxy]-methyl}piperidine-1-carboxylate;
tert-Butyl 4-[({5-[4-(acetylamino)phenyl]pyrimidin-2-yl}oxy)methyl]-piperidine-1-carboxylate;
tert-Butyl 4-{[(5-{4-[(diethylamino)carbonyl]phenyl}pyrimidin-2-yl)oxy]-methyl}piperidine-1-carboxylate;
tert-Butyl 4-[({5-[4-(aminocarbonyl)phenyl]pyrimidin-2-yl}oxy)methyl]-piperidine-1-carboxylate;
tert-Butyl 4-{[(5-{4-[(methylsulfonyl)amino]phenyl}pyrimidin-2-yl)oxy]-methyl}piperidine-1-carboxylate;
tert-Butyl 4-[({5-[4-(morpholin-4-ylcarbonyl)phenyl]pyrimidin-2-yl}oxy)-methyl]piperidine-1-carboxylate;
tert-Butyl 4-[(f{5-[4-(aminosulfonyl)phenyl]pyrimidin-2-yl}oxy)methyl]-piperidine-1-carboxylate;
tert-Butyl 4-{[(5-{4-[(2-hydroxyethyl)sulfonyl]phenyl}pyrimidin-2-yl)oxy]-methyl}piperidine-1-carboxylate;
tert-Butyl 4-[({5-[4-(morpholin-4-ylsulfonyl)phenyl]pyrimidin-2-yl}oxy)-methyl]piperidine-1-carboxylate;
tert-Butyl 4-{[(5-{4-[(2,5-dioxoimidazolidin-1-yl)methyl]phenyl}pyrimidin-2-yl)oxy]methyl}piperidine-1-carboxylate;
tert-Butyl 4-{[(5-{4-[(dimethylamino)sulfonyl]phenyl}pyrimidin-2-yl)oxy]-methyl}piperidine-1-carboxylate;
tert-Butyl 4-[({5-[4-({[2-(dimethylamino)ethyl]amino}carbonyl)phenyl]-pyrimidin-2-yl}oxy)methyl]piperidine-1-carboxylate;
tert-Butyl 4-[({5-[4-(aminocarbonyl)-3-fluorophenyl]pyrimidin-2-yl}oxy)-methyl]piperidine-1-carboxylate;
Isopropyl 4-[({5-[4-(methylsulfonyl)phenyl]pyrimidin-2-yl}amino)methyl]-piperidine-1-carboxylate;
Ethyl 4-[({5-[4-(methylsulfonyl)phenyl]pyrimidin-2-yl}amino)methyl]-piperidine-1-carboxylate;
N-{[1-(3,3-dimethylbutanoyl)piperidin-4-yl]methyl}-5-[4-(methylsulfonyl)-phenyl]pyrimidin-2-amine;
tert-Butyl 4-[({5-[4-(methylsulfonyl)phenyl]pyrimidin-2-yl}amino)methyl]-piperidine-1-carboxylate;
Benzyl 4-[({5-[4-(methylsulfonyl)phenyl]pyrimidin-2-yl}amino)methyl]-piperidine-1-carboxylate;
tert-Butyl 4-({[5-(4-{[(2R)-2-(hydroxymethyl)pyrrolidin-1-yl]carbonyl}-phenyl)pyrimidin-2-yl]oxy}methyl)piperidine-1-carboxylate;
tert-Butyl 4-{[(5-{4-[(2,5-dioxopyrrolidin-1-yl)methyl]phenyl}pyrimidin-2-yl)oxy]methyl}piperidine-1-carboxylate;
tert-Butyl 4-{[(5-{4-[(4-methylpiperazin-1-yl)carbonyl]phenyl}pyrimidin-2-yl)oxy]methyl}piperidine-1-carboxylate;
tert-Butyl 4-({[5-(3,5-difluoro-4-hydroxyphenyl)pyrimidin-2-yl]oxy}methyl) -piperidine-1-carboxylate;
tert-Butyl 4-({[5-(4-{[3-(dimethylamino)pyrrolidin-1-yl]carbonyl}phenyl) -pyrimidin-2-yl]oxy}methyl)piperidine-1-carboxylate;
tert-Butyl 4-{[(5-{3-fluoro-4-[(propylamino)carbonyl]phenyl}pyrimidin-2-yl)-oxy]methyl}piperidine-1-carboxylate;
tert-Butyl 4-{[(5-{4-[(3-hydroxypyrrolidin-1-yl)carbonyl]phenyl}pyrimidin-2-yl)oxy]methyl}piperidine-1-carboxylate;
tert-Butyl 4-[({2-[4-(methylsulfonyl)phenyl]pyrimidin-5-yl}methyl)amino]-piperidine-1-carboxylate; and
tert-Butyl 4-[methyl({2-[4-(methylsulfonyl)phenyl]pyrimidin-5-yl}methyl)-amino]piperidine-1-carboxylate.

21. A method for the treatment or prophylaxis of a disorder relating to GPR119 activity which comprises administering to a mammal, including man, in need of such treatment an effective amount of a compound according to claim 1.

22. The method according to claim 21, wherein said disorder relating to GPR119 activity is selected from the group consisting of Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

23. A pharmaceutical formulation containing a compound according to claim 1 as active ingredient in combination with a pharmaceutically acceptable diluent or carrier.

24. A method for the treatment or prophylaxis of a disorder relating to GPR119 activity which comprises administering to a mammal, including man, in need of such treatment an effective amount of a compound according to claim 1 in combination with a DPP-IV inhibitor.

25. The method according to claim 24, wherein said disorder relating to GPR119 activity is selected from the group consisting of Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

26. The pharmaceutical formulation according to claim 23 which in addition comprises a DPP-IV inhibitor.
